(19) United States
(12) Patent Application Publication
(10) Pub. No.: US 2026/0053874 A1
(43) Pub. Date: Feb. 26, 2026

(54) COMPOSITIONS AND METHODS TO IMPROVE SEQUESTRATION OF MYCOTOXINS AND THEIR USE IN DETOXIFICATION OF ANIMAL FEED AND ANIMAL PRODUCTION SYSTEMS

(71) Applicant: Alltech, Inc., Nicholasville, KY (US)

(72) Inventors: Alexandros Yiannikouris, Lexington, KY (US); Rebecca A. Timmons, Lexington, KY (US); Ursula A. Fox, Lexington, KY (US); Joshua Perry, Mount Sterling, KY (US)

(21) Appl. No.: 19/304,450

(22) Filed: Aug. 19, 2025

Related U.S. Application Data
(60) Provisional application No. 63/685,555, filed on Aug. 21, 2024.
(60) Provisional application No. 63/759,367, filed on Feb. 17, 2025.

(51) Int. Cl. A61K 36/064; A61K 33/06; A61K 35/00; A61K 35/68; A61K 35/741; A61K 35/742; A61K 35/747; A61K 36/02; A61K 36/03; A61K 36/04; A61K 36/05; A61K 36/062; A61P 31/10

(52) U.S. Cl. CPC A61K 36/064; A61K 33/06; A61K 35/68; A61K 35/741; A61K 35/742; A61K 35/747; A61K 36/02; A61K 36/03; A61K 36/04; A61K 36/05; A61K 36/062; A61P 31/10; A61K 2035/115

(57) ABSTRACT

The present disclosure relates to compositions and methods comprising a yeast cell wall extract and a clay material admixed to a proteinaceous and/or cell wall extract(s) from bacterial or fungal fermentation from amino acid production. In another embodiment, the present compositions comprise a yeast cell wall extract, a clay material, and an algal material admixed to a proteinaceous and/or cell wall extract(s) from bacterial or fungal fermentation from amino acid production. This disclosure also relates to methods of utilizing the same to sequester and/or adsorb mycotoxins internal or external to an animal, thereby reducing and/or preventing the toxic and harmful effects of mycotoxin contamination and/or infection in animals. Specifically, application of the present methods and compositions are found in the dietary administration and/or co-administration of the present compositions to animals in animal feed and/or animal production systems, as well as in therapeutic, prophylactic, and research applications.

### CROSS-REFERENCE TO RELATED APPLICATIONS

This nonprovisional application claims the benefit and priority, under 35 U.S.C. § 119 (e) and any other applicable laws or statutes, to U.S. Provisional Patent Application Ser. No. 63/685,555 filed on Aug. 21, 2024 and to U.S. Provisional Patent Application Ser. No. 63/759,367 filed on Feb. 17, 2025, the entire disclosures of all of which are hereby expressly incorporated herein by reference.

### TECHNICAL FIELD OF THE INVENTION

The present invention relates to methods and/or compositions, when admixed to feed materials, incorporated directly into pelleted blocks, and/or administered directly, top-dressed into feed, that possess the ability to sequester mycotoxins and limit or prevent their absorption during their passage in the digestive tract of animals. In particular, the invention relates to the use of compositions in combination with yeast cell wall-containing preparations amended with a proteinaceous extract from a bacterial, or fungal fermentation process, either in liquid or solid state, used in amino acid production. These novel compositions and methods have shown synergistic properties in adsorption, sorption, sequestration, and binding of mycotoxins inside the digestive tract of animals. Consequently, the present compositions and methods work to decrease the absorption and bioavailability of toxins to animals.

### BACKGROUND

Mycotoxins are naturally occurring toxins produced by various fungal or mold/mould species, affecting a wide array of agricultural products and crops that may be commercial commodities. Mycotoxins are secondary fungal metabolites primarily from a genera, such as Aspergillus, Penicillium, and Fusarium, though not limited to those. Mycotoxins are commonly found in cereal grains, forages, and stored animal feed ingredients, both before and after harvest and/or processing.

Fungal spores typically encounter agricultural crops, leading to its contamination or infection. The production of mycotoxins in plants is influenced by a combination of biotic and abiotic factors, including the plant's cultivar, health status prior to harvest, weather patterns and conditions, geographical location and topography, agricultural practices, agronomical factors, harvesting techniques, delays, hydrothermal, watering, or sun conditions. Mycotoxin production and occurrence is also affected by the processes employed when animal feed is produced, transformed, processed, transported, and/or stored.

Fungal spores are widespread in the environment, and they thrive in various ecological niches by metabolically breaking down organic matter under particular environmental and biological conditions. Both biotic and abiotic stressors can prompt fungal species to produce secondary metabolites, such as mycotoxins. Fungal growth is influenced by one or more physicochemical factors, which vary depending on the fungal strain. These physicochemical factors leading to fungal growth include one or more of the availability of free water (aw), temperature, oxygen-levels or concentrations, substrate characteristics, flowering stage, and/or pH conditions. Mycotoxins can be produced both before harvest and during the post-harvest storage phase. Animals, including rodents, birds, and insects, can contribute to contamination by physically damaging plants, which creates entry points for fungal spores to infect and/or contaminate the plant or crop.

Fungi that produce toxins can be classified into two main groups. A first fungal group directly contaminates plants and agricultural commodities, such as nuts, fruits, and/or vegetables, while they are still in the field. A second fungal group develops on a stored animal feed, a feed or food ingredient or component (like silages), or during certain processes of food or animal feed ingredients (such as fermentation, nixtamalization, or separations).

Fungi can also affect other organic materials, including bedding, clothing, water and/or food sources where animals may come in contact. Additionally, the global trade of animal feed ingredients and components increases the likelihood of grain blends containing various combinations of mycotoxins, leading to unexpected and sometimes rare fungal contamination patterns in specific diets.

Certain fungal species produce mycotoxins only under specific conditions of moisture, temperature, or oxygen levels, and these conditions may vary between species. Some or different fungi or fungal strains are also capable of producing the same mycotoxin(s). The impact of mycotoxins can range from severe effects-such as causing death or distinct diseases to more subtle consequences, like weakening the animal immune system without obvious symptoms or signs of infection, acting as allergens or irritants, or sometimes having no noticeable effect on animals or humans at all.

In fact, it wasn't until 1960, however, that the scientific community recognized the dangers of mycotoxins, following the devastating Turkey X Syndrome outbreak in Britain, which killed over 100,000 turkeys. Since then, advances in detection methods (such as chromatography and mass spectrometry) have led to the identification of thousands of fungal metabolites, with fewer than 100 metabolites known to cause harmful effects to plants and crops, animals, and/or humans. Of those, only about 30 metabolites are known to have varying toxicological impacts on plants and crops, animals, and/or humans.

Agronomic practices, such as tilling or the uncontrolled use of fungicides, can also influence the bio-recycling of mycotoxins and their occurrence. As a result, mycotoxins can be present in plant-based foods and feeds, and they may become concentrated in certain processed feed products. For instance, distiller's dried grains with solubles (DDGS) can be a potential source of mycotoxins, as they survive the ethanol production process and may become concentrated up to three times in the residual matrix of the DDGS.

Identifying, preventing and/or mitigating mycotoxin contamination in the food and feed supply chain requires a comprehensive management, including the implementation of critical control points to ensure quality control and—reduce the prevalence of mycotoxins. However, mycotoxin contamination often goes undetected due to one or more factors, such as limited sampling plans, limited access to multiplex detection techniques, and/or the inherent variability of the methods used for detection and quantification of mycotoxins, as well as their adaptability to a wide range of different feed matrices used in animal feed production.

Mycotoxins are typically small molecules, usually under 1,000 Da, and belong to various categories such as polyketides, coumarin derivatives, cyclic terpenes, sesquiterpene epoxides, macrolides, sphingolipids, amino acid derivatives, cyclopeptides, and alkaloids. Many of these toxins are highly stable at both elevated temperatures and varying pH values, making them resistant to elimination from food through physical or chemical processes.

Mycotoxins, such as aflatoxins, and including aflatoxin B1 and aflatoxin M1, are classified as group 1 carcinogens by the International Agency for Research on Cancer (IARC). As a result, maximum permissible limits for aflatoxins in food and feed have been established in various regions, such as Europe (Directive 2002/32/EC) and the United States (FDA Guidance for Industry, 2020). Other mycotoxins are also under regulatory scrutiny, with specific concentration thresholds set, each with distinct properties and toxicological effects that can present significant risks to both human and animal health.

Key mycotoxins of concern include aflatoxins, primarily produced by Aspergillus species like A. flavus and A. parasiticus, and commonly found in crops such as peanuts, maize, and tree nuts. Another major category of concern is trichothecenes, which are produced by various Fusarium species. This group of problematic mycotoxins includes among others, deoxynivalenol (DON), T-2 toxin, and HT-2 toxin. These mycotoxins can lead to a range of harmful effects, including the inhibition of protein synthesis and immune suppression in animals and humans. There are additional mycotoxins that are also known to have negative effects on humans and animals.

For example, ochratoxins are produced by an Aspergillus and/or Penicillium species, Ochratoxin A is the most common variant. This mycotoxin is linked to nephrotoxicity (i.e., kidney damage) and carcinogenic effects in animals and/or humans.

Zearalenone synthesized by Fusarium species, particularly F. graminearum, has estrogenic properties and can lead to reproductive disorders in animals, and possibly humans as well.

Fumonisins are primarily produced by Fusarium verticillioides and other related fungal species. These mycotoxins are associated with diseases, such as equine leukoencephalomalacia and are often found in maize and its derivatives products (e.g., maize-containing animal or bird feeds).

Ergot alkaloids are produced by a Claviceps species, particularly C. purpurea. These mycotoxins are associated with ergotism, a condition that affects the nervous system and can cause blood vessel constriction (e.g., vasoconstriction) in animals and humans.

Patulin is another mycotoxin generated by various Penicillium and Aspergillus species. Patulin is also found in feedstuffs, and is also commonly found in fruits and fruit-based products. It has been linked to gastrointestinal irritation and potential genotoxic effects in animals and humans.

While certain mycotoxins present significant risks to both animal and/or human health, especially at acute levels, and cause issues in animal feed production, including productivity losses from chronic exposure, there are a few practical solutions currently available for assessing mycotoxin exposure risks and effectively mitigating mycotoxin contamination.

For example, adding large amounts, from about 2 kg/T to about 10 kg/T inclusion rate in the diet, of various clay compositions to animal feed has shown limited effectiveness in sequestering certain mycotoxins within the animal's gastrointestinal tract. However, clays can also inhibit and/or hinder the absorption of vital nutrients, such as vitamins, minerals, and amino acids, reducing the overall nutritional value of any animal feed diet in which they are comprised. Additionally, as mining and/or industrially processed products, they could be prone to environmental and industrial toxins contamination (i.e., heavy metals, PCB/dioxins, radionuclei, etc.). Since clays are inert materials that require substantial quantities to be effective, they remain undigested in the animal gut and/or digestive system, and are excreted in the animal's feces. This excretion includes some of the mycotoxins that the clays have adsorbed or sequestered, contributing to environmental pollution and safety hazard when discarded in animal feces left in the environment, such as on a farm on in a field.

The shortcomings of using clays alone as sequestering agents prompted the development of new strategies, such as combining yeast cell wall extracts with clays. For instance, previous technologies involved concentrated, modified yeast cell wall extracts paired with small amounts of clay, which was thought to provide a more efficient way to protect both animals and humans from the harmful effects of mycotoxins. Other prior art included a more technological interfacing of yeast cell wall and clay producing an innovative interlaced material aiming at improving sorption efficacy toward, but not limited to, aflatoxin B1 and zearalenone. Further advancements also led to the creation of tailored-made synthetic adsorbents designed to target specific mycotoxins or groups of mycotoxins (i.e., molecularly imprinted polymers).

Despite these advancements in addressing mycotoxin contamination, there is still room for improvement in the functionality and efficacy of yeast cell wall extract-based adsorbents, particularly in targeting newly emerging mycotoxins or expanding the adsorption capacity toward commonly known mycotoxins. The present methods and compositions for mycotoxin detection and/or mitigation expand the efficacy of current products and protects animals and crops against different and broader categories of mycotoxins. Specifically, the present innovations are necessary to enhance current mycotoxin management strategies and more effectively mitigate the risks of harm posed by mycotoxins contamination to animals, animal feed, and animal production systems, as well as humans.

As highlighted earlier, the ubiquitous and widespread presence of mycotoxins, particularly deoxynivalenol (DON), along with other lesser-studied toxins like fusaric acid (FA) and other emerging mycotoxins, such as enniatins or penicillic acid, and the co-occurrence of various other mycotoxins often averaging 5 to 7 different mycotoxins at a time, has been documented in many modern feed ingredient worldwide surveys. If the contamination of food crops has been estimated by the Food and Agricultural Organization (FAO) to affect around 25% of crops worldwide, with advancements in analytical techniques and instrumentation, alongside more adequate sampling plans, mycotoxins are now being detected in 60 to 80% of crops, revealing new patterns of contamination and the presence of variants like “masked” mycotoxins.

Although in regions like Europe and the United States, only aflatoxin B1 (AFB1) in feeds and aflatoxin M1 (AFM1) in milk are strictly regulated, and recommendations are provided for mycotoxins such as DON, zearalenone (ZEA), ochratoxin A (OTA), patulin, and fumonisins in animal production, there remains a considerable risk of animals consuming mycotoxin-contaminated feed. Moreover, current regulations do not account for the simultaneous presence of multiple mycotoxins, which may lead to additive, coalistic, or synergistic effects, further exacerbating mycotoxin's impact, as demonstrated in both in vitro and in vivo studies.

The risks posed to animals by mycotoxins in animal feed and mycotoxin exposure during animal production can be summarized in two main concerns. The first (i) is animal intoxication. While acute animal poisoning is uncommon, chronic exposure to mycotoxins is more frequent and difficult to diagnose. Mycotoxin intoxications of animals generally present nonspecific clinical signs, and veterinarians often consider this diagnosis only after other treatments fail, except in the cases of ZEA or T-2 toxin presence, which display more obvious pathological signs. The second (ii) concern is the transfer of toxins to animal by-products. Mycotoxins can also contaminate animal products such as milk, meat, and eggs, which also enter the human food chain. These products may contain both the parent mycotoxins and their metabolites, whose toxicity is not always well understood, but believed to have dangerous effects for both animals and humans.

Given that completely eliminating mycotoxins from feed ingredients, even with stringent control and protections, is not technologically feasible to date, there has been an increasing demand for products that can deactivate mycotoxins in the animal's digestive tract when added to contaminated feed. This strategy aims to reduce the harmful effects of mycotoxins on animals and enhance the safety of animal by-products. Therefore, deactivating mycotoxins before they enter the bloodstream of animals is considered a primary and effective strategy to address natural feed contamination with mycotoxins, which is the goal of the present inventive compositions and methods.

Processed clays, particularly hydrated sodium calcium aluminosilicates (HSCAS), have been shown to bind AFB1 in vitro when added at appropriate levels, often from 2.0 to 10.0 kg/T of inclusion rate equivalent in the diet, and to a lesser extent in vivo. However, clays like HSCAS, zeolites, and/or bentonites, have shown limited efficacy in binding mycotoxins produced by other fungi. However, clays, including HSCAS, zeolites, and bentonites, have shown limited efficacy in binding other mycotoxins at inclusion levels below 1% in feed and could interact with the diet nutrient density.

As a result, research has increasingly focused on identifying organic compounds that can efficiently adsorb a wider range of naturally co-occurring mycotoxins in mixed feeds. Key objectives include achieving effective toxin sequestration at low inclusion levels, from about 0.05 or lower to about 4.0 kg/T equivalent in the diet, and ensuring biodegradability, which would allow for flexible formulations while minimizing environmental impact.

Mycotoxin inactivators can be categorized based on their mode of action. One group comprises agents that chemically interact with mycotoxins, preventing their absorption in the animal's digestive system (often referred to as sorbents, adsorbents, sequestrants, or binders). These agents are further divided into organic and inorganic categories. While inorganic adsorbents comprise clays, the organic inactivators of mycotoxins, such as yeast cell wall extracts, have demonstrated effectiveness in mitigating mycotoxin challenges both in vitro and in vivo. They also comprise mycotoxin-degrading enzymes produced by microbial strains, such as bacteria and yeasts.

Early studies explored the effects of organic agents, such as live yeast cultures, on aflatoxicosis in poultry, which showed improvements in hatchability, increased body weight gain in broilers, and enhanced immune responses. In vitro research confirmed that aflatoxins could be bound to purified yeast cell walls in a dose-dependent manner. Further studies demonstrated that the yeast cell wall of Saccharomyces cerevisiae could bind a range of mycotoxins, including ZEA (66.7%), fumonisins (67.0%), DON (12.6%), OTA (12.5%), citrinin (18.4%), T2 (33.4%), and diacetoxyscirpenol (DAS, 12.7%).

In addition, Applicant conducted fractionation studies, previously published in scientific literature, of different yeast strains, isolating three components: 1) total cell wall, 2) alkali-soluble glucan, and 3) alkali-insoluble glucan. These studies revealed that the adsorption of mycotoxins is driven by the carbohydrate fraction of yeast cell walls, particularly one or more glucan polymers (“glucans”) arranged in a fibrillar network. These glucans of a yeast cell wall play a key role in sequestering mycotoxins, such as AFB1, ZEA, DON, and patulin.

X-ray diffraction analysis showed that these mycotoxins form complexes with the glucan chains of the yeast cell wall. Stability between the mycotoxins and yeast cell wall glucans is largely attributed to weak interactions, like hydrogen bonds and van der Waals forces. While individually weak, these interactions collectively contribute to the stability of a yeast cell wall-mycotoxin complex, resembling covalent bonds and making it more resilient and resistant to environmental changes. For example, β-(1,3)-D-glucans, branched with β-(1,6)-D-glucans, form a helix-shaped structure that provides multiple binding sites matching the geometric and steric properties of the mycotoxins, further enhancing stability of the yeast cell wall-mycotoxin complex through van der Waals interactions and hydrogen bonding.

These findings, alongside studies on carbohydrate conformation, suggest that β-D-glucans from S. cerevisiae and other fungi, along with their specific stereochemical conformation, are effective in binding a wide range of mycotoxins, without interfering with nutrient interactions. Thus, β-D-glucans of yeast cell wall makes these interactions particularly useful in multi-contamination situations. Additionally, β-D-glucans are environmentally friendly due to their biodegradability. These published studies by Applicant confirm that the level and complexity of glucans (e.g., β-D-glucans) enhance and increase mycotoxin binding and stability.

Further in vivo studies of yeast cell wall-based strategies have produced numerous prior publications investigating the protective effects of mycotoxin inactivators, many of which have focused on organic adsorbents. Research has been conducted across various animal species, including poultry, horses, dogs, and fish species to evaluate the effectiveness of these strategies. The present disclosure comprises additional studies to evidence the efficacy of one or more improved mycotoxin sequestering compositions and methods, as described and claimed herein, for reduction and/or removal of mycotoxin contamination in animals, such as cows, cattle, swine, avian species and aquatic species. The present compositions and methods can be applied as feed ingredients, feed additives, and/or immobilized feed ingredients in blocks or boluses, but their use is not limited to these forms.

### SUMMARY

The present disclosure is directed to a co-mixture composition that is configured to interact with a mycotoxin, comprising: a) a yeast cell, including a yeast cell wall or extract therefrom, having about 20 wt % to about 99 wt % of the composition and b) a clay material having about 0.1 wt % to about 10 wt % of the composition, admixed to c) a proteinaceous or cell wall extract from a bacterial fermentation process or a fungal fermentation process having about 0.5 wt % to about 99 wt % of the composition. The co-mixture composition may further comprise an algal material having about 1 wt % to about 99 wt % of the composition.

The yeast cell of the co-mixture composition may be selected from the group consisting of Saccharomyces, Candida, Kluyveromyces, Torulaspora, and a combination thereof. The clay material of the co-mixture composition may be a zeolite, a bentonite, an aluminosilicate, a montmorillonite, a smectite, a kaolinite, an organoclay, a modified clay, or mixtures thereof.

The proteinaceous and/or cell wall extract(s) of the co-mixture composition may be selected from a bacterial strain selected from the group consisting of the genus Corynebacterium, Brevibacterium, Escherichia coli, Enterobacter, Lactobacillus, Pseudomonas, and Bacillus or a fungal strain selected from the group consisting of the genus Aspergillus, Candida, Fusarium, and Saccharomyces that is used in an industrial fermentation process. The proteinaceous or cell wall extract from the bacterial fermentation process or the fungal fermentation process may be dried.

The algal material of the co-mixture composition may belong to the algae cell division Chlorophyceae (green algae), Chromophyta, Chriptophyta, Rhodophyta (red algae), Dinoflagellata (Pyrrophyta), Euglenophyta or from Chlorella, Asterarcys quadricellulare, Aurantiochytrium, and Schizochytrium species.

The mycotoxin interacting with the co-mixture composition may be selected from the group consisting of aflatoxins, ochratoxins, fumonisins, emerging Fusarium mycotoxins, Aspergillus mycotoxins, Penicillium mycotoxins, zearalenone, ergot alkaloids mycotoxins, AAL toxins, acetoxyscirpenediol, acetyldeoxynivalenol, acetylneosolaniol, acetyl T-2 toxin, acetyl HT-2, aflatoxins including aflatoxin B1 and B2 and G1 and G2, aflatoxicol, aflatrem, altenuic acid, alternariol, altertoxin, altersolanols, Alternaria toxins, apicidins, arugosins, asperazines, aspergillic acid, aspergillumarins, asperlicins, aspewentins, aspochalasins, aurofusarin, aurosperones, aurovertins, austalides, austdiol, austamide, austocystin, avenacein, baccharinoids, beauvericin, bentenolide, brevianamide, calonectrin, chaetoglobosin, chevalones, citrinin, citreoviridin, citreoviridinol, cochliodinol, coniochaetons, cytochalasins, cyclosporins, cytochalasins, cyclopiazonic acid, deacetylcalonectrin, decarestrictine, deoxynivalenol, diacetoxyscirpenol, diacetyldeoxynivalenol, destruxins A and B, elymoclavines, enniatins such as enniatins A/A1 and B/B1, ergot toxins and endophytes such as ergine, ergocornine, ergocristine, ergocryptine, ergometrine, ergonine, ergosine, ergotamine, ergovaline, lysergol, lysergic acid, methylergonovine, and related epimers, fructigenines, fumigaclavines, fumagillin, fumiquinazolines, fumitremorgins, fumonisins including fumonisin A1 and B1 and B2 and B3, fusarenon X, fusaric acid, fusarin, fusarielin, fuscofusarin, geodin, geomycins, gliotoxin, griseophenones, griseofulvin, HT-2 toxin, ipomeanine, islanditoxin, isofumigaclavines A and B lateritin, leporisines, lolitrems, lycomarasmine, malformins, marcfortines, maleagrins, maltoryzine, miophytocens, moniliformin, monoacetoxyscirpenol, mycophenolic acid, neosolaniol, nigerapyrones, nivalenol, nordeodeoxynivalenol, NT-1 toxin, NT-2 toxin, ochratoxins such as ochratoxins A and B, oxalic acid, paraherquamide, paspalines, paspalitrems A and B, patulin, paxilline, penicillenol, penicillic acid, penitrems such as penitrem A, phomopsins, PR-toxin, psychrophilins, pyripyropenes, roridins, roritoxins, roquefortines such as roquefortine C, rubratoxin, rubroskyrin, rubrosulphin, rugulosin, satratoxins, scirpentriol, slaframine, solaniol, sporotrichiol, stephacisins, sterigmatocystin, sulochrin, swainsonine, T-2 toxin, tentoxin, terreins, territrems, tetrahydroaltersolanols, triacetoxyscirpendiol, trichothecenes, trichodermin, trichothecin, trichoverrins, trichoverrols, tryptoquivalene, verrucarins, versicolorins, versiconols, verruculogen, viopurpurin, viomellein, viriditoxin, wortmannin, xanthocillin, xanthomegnin, yavanicin, zanones, zearalenols, zearalanones, zearalenone and subfamilies, and/or possible conjugates and metabolites of the aforementioned mycotoxins, and combinations thereof.

The co-mixture composition may have an average adsorption or sequestration rate for one or more mycotoxins ranging from about 33% to about 100%. At least a portion of the co-mixture composition has an improved efficacy to sequester or adsorb one or more mycotoxins over traditional compositions. For example, the co-mixture composition has an increased average adsorption rate for deoxynivalenol (DON) of about 211% or for fusaric acid of about 285%. In addition, the co-mixture composition does not reduce its ability to interact with the mycotoxin when ground, dried, spray dried, vacuum dried, or heated.

The present disclosure is also directed to a method of sequestering one or more mycotoxins in an animal, comprising: a) providing to the animal for feeding or consumption, a composition comprising: i) a yeast cell, including a yeast cell wall or extract therefrom, having about 20 wt % to about 99 wt % of the composition, ii) a clay material having about 0.1 wt % to about 10 wt % of the composition, iii) an optional algal material having about 0 wt % to about 10 wt %, admixed to iv) a proteinaceous or cell wall extract from a bacterial fermentation process or a fungal fermentation process having about 0.5 wt % to about 99 wt % of the composition, and v) an optional carrier having about 0 wt % to about 50 wt % of the composition, b) increasing sequestration or adsorption of the one or more mycotoxins within the gut of the animal, and c) reducing absorption of the one or more mycotoxins in the bloodstream of the animal.

The method may further comprise feeding the composition to the animal in an amount ranging from about 2 g/animal/day to about 50 g/animal/day. Consumption of the composition by the animal may further comprise providing the composition with an organic material selected from the group consisting of an animal feedstuff, a liquid, a water, an animal bedding and an animal clothing. Providing the composition with the animal feedstuff may comprise the composition being at about 0.0125 wt % to about 10 wt % of the animal feedstuff. Alternatively, providing the composition with the animal feedstuff may comprise an inclusion rate of the composition at about 0.125 to about 4.0 kg/T of the animal feedstuff. In addition, grinding, drying, spray drying, vacuum drying, or heating the composition during the method does not reduce its ability to sequester or adsorb the one or more mycotoxins.

The present disclosure is also directed to a mycotoxin-sequestering composition, comprising: a) a yeast cell, including a yeast cell wall or extract therefrom, having about 70 wt % to about 90 wt % of the composition, b) a clay material having about 0.1 wt % to about 4 wt % of the composition, c) an algal material having about 1 wt % to about 10 wt % of the composition, all admixed to) a dried proteinaceous or cell wall extract from a bacterial fermentation process or a fungal fermentation process having about 10 wt % to about 30 wt % of the composition, and e) a carrier having about 1 wt % to about 50 wt % of the composition, wherein at least a portion of the mycotoxin-sequestering composition has an improved efficacy over traditional compositions to sequester or adsorb one or more mycotoxins present on or within the animal. The mycotoxin-sequestering composition of may have an increased average adsorption rate for deoxynivalenol (DON) of about 211% or for fusaric acid of about 285% over traditional or historical compositions.

### BRIEF DESCRIPTION OF THE DRAWINGS

FIG. 1 is a spider-web graphical representation of the percent (%) of mycotoxin adsorption using a kinetic of interaction between the present composition, comprising a yeast cell wall extract and clay material admixed to a proteinaceous extract from bacterial fermentation from amino acid production, with 6 titers ranging from 500 to 5000 ng/ml of an iso-mixture of 8 mycotoxins at pH 3.0;

FIG. 2 is a spider-web graphical representation of the percent (%) of mycotoxin adsorption using a kinetic of interaction between the present composition, comprising a yeast cell wall extract and clay material admixed to a proteinaceous extract from bacterial fermentation from amino acid production, with 6 titers ranging from 500 to 5000 ng/mL of an iso-mixture of 8 mycotoxins at pH 3.0;

FIG. 3 is a histogram of the percentage (%) of cell viability relative to the control of IPEC-J2 cells in the presence of 9 different titers of deoxynivalenol (0.5 to 10.0 mg/ml or ppm) with (orange bars) or without (blue bars) the use of the present invention at an inclusion rate of 0.2% (w/v);

FIG. 4 is a histogram of the percentage (%) of cell viability relative to the control of IPEC-J2 cells in the presence of 9 different titers of deoxynivalenol and fusaric acid iso-mixture (0.5 to 10.0 mg/mL or ppm) with (orange bars) or without (blue bars) the use of the present invention at an inclusion rate of 0.2% (w/v);

FIG. 5 is a spider-web graphical representation of the percent (%) of mycotoxin adsorption between the present composition comprising (1) a yeast cell wall extract and clay material admixed to bacterial biomass rich in protein, (2) a yeast cell wall extract, a clay material and an algal material admixed to bacterial biomass rich in protein, (3) a yeast cell wall extract and clay material mixture, and (4) a yeast cell wall extract and clay material admixed to algal material, and a mycotoxin isomixture at a concentration of 1.0 μg/mL that was evaluated at pH 3.0;

FIG. 6A is a bar graph showing a feed conversion ratio of broiler chicken challenged with natural Fusarium mycotoxins (DON) with or without the supplementation of a yeast cell wall extract and clay material admixed to bacterial biomass rich in protein used at 3 inclusion rates (1.0, 2.0, 4.0 kg/T) during an experimental period of 22-28 days. Different letters above the bars indicate significant differences between treatments, p≤0.05 (ANOVA followed by Tukey's test);

FIG. 6B is a bar graph showing a feed conversion ratio of broiler chicken challenged with natural Fusarium mycotoxins (DON) with or without the supplementation of a yeast cell wall extract and clay material admixed to bacterial biomass rich in protein used at 3 inclusion rates (1.0, 2.0, 4.0 kg/T) during an entire experimental period of 10-41 days. Different letters above the bars indicate significant differences between treatments, p≤0.05 (ANOVA followed by Tukey's test);

FIG. 7A is a bar graph showing blood calprotectin corrected for creatinine levels in broiler chicken challenged with natural Fusarium mycotoxins (DON) with or without the supplementation of a yeast cell wall extract and clay material admixed to bacterial biomass rich in protein used at 3 inclusion rates (1.0, 2.0, 4.0 kg/T). Different letters above the bars indicate significant differences between treatments, p≤0.05 (ANOVA followed by Tukey's test);

FIG. 7B is a bar graph showing cecal calprotectin in broiler chicken challenged with natural Fusarium mycotoxins (DON) with or without the supplementation of a yeast cell wall extract and clay material admixed to bacterial biomass rich in protein used at 3 inclusion rates (1.0, 2.0, 4.0 kg/T). Different letters above the bars indicate significant differences between treatments, p≤0.05 (ANOVA followed by Tukey's test);

FIG. 8A is a bar graph showing short chain volatile fatty acid, butyrate, levels (mmol/kg) in broiler chicken challenged with natural Fusarium mycotoxins (DON) with or without the supplementation of a yeast cell wall extract and clay material admixed to bacterial biomass rich in protein used at an inclusion rate of 4.0 kg/T. Different letters above the bars indicate significant differences between treatments, p<0.05 (ANOVA followed by Tukey's test);

FIG. 8B is a bar graph showing the percent (%) of short chain volatile fatty acid (SCFAs), butyrate, levels (mmol/kg) of unchallenged animals compared to broiler chicken challenged with natural Fusarium mycotoxins (DON) with or without the supplementation of a yeast cell wall extract and clay material admixed to bacterial biomass rich in protein used at an inclusion rate of 4.0 kg/T. Different letters above the bars indicate significant differences between treatments, p≤0.05 (ANOVA followed by Tukey's test);

FIG. 9 is a bar graph showing butyrate kinase and butyryl-CoA acetate transferase encoding genes evaluated in cecal samples collected from broiler chicken challenged with natural Fusarium mycotoxins (DON) with or without the supplementation of a (1) yeast cell wall extract and clay material admixed to bacterial biomass rich in protein used at an inclusion rate of 4.0 kg/T. Different letters above the bars indicate significant differences between treatments, p≤0.05 (ANOVA followed by Tukey's test);

FIG. 10 is a bar graph showing deoxynivalenol (DON) concentrations in blood corrected with 13C-isotypically labeled internal standards in broiler chicken at day-28 and day-41 when challenged with natural Fusarium mycotoxins (DON) with or without the supplementation of a yeast cell wall extract and clay material admixed to bacterial biomass rich in protein used at 3 inclusion rates (1.0, 2.0, 4.0 kg/T). Different letters above the bars indicate significant differences between treatments, p≤0.05 (ANOVA followed by Tukey's test); and

FIG. 11 is a bar graph showing a relative quantification (expressed in response counts) of a phase II metabolite of deoxynivalenol, deoxynivalenol-sulfate (DON3S), in blood collected at day-28 and -41 in broiler chicken challenged with natural Fusarium mycotoxins (DON) with or without the supplementation of a yeast cell wall extract and clay material admixed to bacterial biomass rich in protein used at 2 inclusion rates (2.0 & 4.0 kg/T). Different letters above the bars indicate significant differences between treatments, p≤0.05 (ANOVA followed by Tukey's test).

### DETAILED DESCRIPTION

The present invention described herein relates to one or more compositions and/or methods for an improved yeast cell wall-based product offering unexpected technical benefits and technological advancements when combined with a proteinaceous extract produced through bacterial or fungal fermentation from an amino acid production process. The methods and compositions described herein fall under the first category of organic mycotoxin inactivators that are designed to sequester and/or adsorb mycotoxins. These compositions and methods sequester and/or adsorb mycotoxins, prevent mycotoxins from migrating from an animal's digestive tract into their bloodstream, and/or deactivate mycotoxins before they enter the bloodstream of an animal, helping to enhance the clearance of the mycotoxins from the animal's system and reducing their toxic effects on the animal.

In some embodiments, the present invention provides a novel co-mixture composition comprising: 1) a yeast cell wall extract(s) and 2) a clay material(s) admixed to 3) a proteinaceous and/or cell wall extract(s) from bacterial or fungal fermentation from amino acid production, also known as a bacterial biomass rich in protein. The present invention may be in the presence or absence of one or more carrier(s).

In some embodiments comprising a carrier, the amount of carrier is from about 0 wt % to about 50 wt % of the present composition, including any specific or variety of ranges of weight percent (wt %) comprised therein. In further embodiments, the amount of carrier is from about 0 wt % to about 40.0 wt %, about 0.0 wt % to about 30.0 wt %, about 0.0 wt % to about 20.0 wt %, about 0.0 wt % to about 10.0 wt %, about 1.0 wt % to about 50.0 wt %, about 1.0 wt % to about 40.0 wt %, about 1.0 wt % to about 30.0 wt %, about 1.0 wt % to about 20.0 wt %, about 1.0 wt % to about 10.0 wt %, about 1.0 wt % to about 5.0 wt %, about 5.0 wt % to about 45.0 wt %, about 5.0 wt % to about 30.0 wt %, about 5.0 wt % to about 20.0 wt %, about 5.0 wt % to about 10.0 wt %, and about 5.0 wt % to about 50.0 wt % of the present composition

In some embodiments, the yeast cell wall extract of the present invention is selected from Saccharomyces, Candida, Kluyveromyces, Torulaspora, and/or combinations thereof. In some Saccharomyces embodiments, the yeast cell wall extract is from Saccharomyces cerevisiae. In other embodiments, the Saccharomyces (e.g., cerevisiae) yeast cell wall extract may be combined with cell wall extracts of other yeasts, such as those noted above, and any others.

In some embodiments, the effective amount of yeast cell wall extract is from between about 20.0 wt % to about 99.0 wt % of the present composition, including any specific or variety of ranges of weight percent (wt %) comprised therein. In other embodiments, the effective amount of yeast cell wall extract is from between about 40.0 wt % to about 60.0 wt % or from between about 70.0 wt % to about 90.0 wt % of the present composition. In further embodiments, the effective amount of yeast cell wall extract is from about 30.0 wt % to about 90.0 wt %, about 40.0 wt % to about 80.0 wt %, about 50.0 wt % to about 70.0 wt %, about 20.0 wt % to about 50.0 wt %, about 25.0 wt % to about 50.0 wt %, and about 75.0 wt % to about 95.0 wt % of the present composition

In some embodiments, the proteinaceous and/or cell wall extract(s) is collected as a material from a bacterial or fungal fermentation used for the production of amino acids, also known as a bacterial or fungal biomass rich in protein, respectively. In some embodiments, the proteinaceous and/or cell wall extract(s) is collected as a waste material from a bacterial or fungal fermentation process used for the production of amino acids. In other embodiments, the proteinaceous and/or cell wall extract(s) is collected as a residual material from a bacterial or fungal fermentation used for the production of amino acids.

In some embodiments, the proteinaceous extract and not the cell wall extract, is collected as a material from a bacterial or fungal fermentation process used for the production of amino acids. In other embodiments, the cell wall extract and not the proteinaceous extract, is collected as a material from a bacterial or fungal fermentation process used for the production of amino acids. In further embodiments, the proteinaceous and the cell wall extract are collected as a material from a bacterial or fungal fermentation process used for the production of amino acids.

In further embodiments, the proteinaceous and/or cell wall extract(s) is collected as a material from a bacterial fermentation used for the production of amino acids and not a fungal fermentation used for the production of amino acids. In additional embodiments, the proteinaceous and/or cell wall extract(s) is collected as a material from a fungal fermentation used for the production of amino acids and not a bacterial fermentation used for the production of amino acids. In further embodiments, the proteinaceous and the cell wall extract are collected as a material from a bacterial and a fungal fermentation process used for the production of amino acids. For example, such an embodiment of the present composition comprising the proteinaceous and the cell wall extracts from both a bacterial and a fungal fermentation process used for the production of amino acids may comprise about 5 wt % to about 50% of the bacterial biomass rich in protein, including any specific or range of percentages comprised therein (e.g., about 10 wt % as demonstrated in composition J), as well as about 5 wt % to about 50% of the fungal biomass rich in protein, including any specific or range of percentages comprised therein (e.g., about 35 wt % as demonstrated in composition J).

In some embodiments, the fungal fermentation extract used to produce amino acids in the present composition is selected from one or more fungal strains. Fungal strains utilized to provide fungal fermentation extract or the fungal biomass rich in protein for the present invention belong to the genus Aspergillus, Candida, Fusarium, and Saccharomyces, or combinations thereof.

In some embodiments, the bacteria fermentation extract used for amino acid production is selected from one or more bacterial strains. Bacterial strains utilized to provide bacterial fermentation extract for the present invention belong to the genus Corynebacterium, Brevibacterium, Escherichia coli, Enterobacter, Lactobacillus, Pseudomonas, Bacillus, other bacterial strains used in industrial fermentation processes, or combinations thereof. In other embodiments, the bacterial strain is from the genus Corynebacterium or Brevibacterium. In further embodiments, the bacterial strain is Corynebacterium glutamicum.

In some embodiments, the effective amount of fungal or bacterial biomass rich in protein (i.e., the proteinaceous and/or cell wall extract(s) is collected as a material from a bacterial or fungal fermentation used for the production of amino acids) is from between about 0.5 wt % to about 99.0 wt % of the present composition, including any specific or variety of ranges of weight percent (wt %) comprised therein. In other embodiments, the effective amount of fungal or bacterial biomass rich in protein is greater than 99 wt % of the present composition. In further embodiments, the effective amount of bacterial proteinaceous and/or cell wall extract(s) is from between about 20.0 wt % to about 55.0 wt % or from between about 10 wt % to about 30 wt % of the present composition. In further embodiments, the effective amount of bacterial proteinaceous and/or cell wall extract(s) is from about 1.0 wt % to about 10.0 wt %, about 5.0 wt % to about 25.0 wt %, about 20.0 wt % to about 75.0 wt %, about 30.0 wt % to about 60.0 wt %, about 20.0 wt % to about 50.0 wt %, about 15.0 wt % to about 25.0 wt %, about 20.0 wt % to about 80.0 wt %, and about 75.0 wt % to about 95.0 wt % of the present composition

In some embodiments, the present invention provides for one or more clay or clay-based materials (“a clay”). In some embodiments, a clay of the present composition includes, but is not limited to a zeolite, a bentonite, an aluminosilicate, a montmorillonite, a smectite, a kaolinite, an organoclay, a modified clay, and/or mixtures and combinations thereof. In some embodiments, the clay is an aluminosilicate clay.

In some embodiments, the effective amount of the clay is from about 0.1 wt % to about 20.0 wt % of the present composition, including any specific or variety of ranges of weight percent (wt %) comprised therein. In other embodiments, the effective amount of the clay in the present composition is from between about 5.0 wt % to about 25.0 wt %, about 5.0 wt % to about 20.0 wt %, from between about 1.0 wt % to about 10.0 wt %, or is from between about 0.1 wt % to about 2.0 wt %. In further embodiments, the effective amount the clay is from about 0.1 wt % to about 3.0 wt %, about 0.1 wt % to about 4.0 wt %, about 0.1 wt % to about 5.0 wt %, about 0.1 wt % to about 6.0 wt %, about 0.1 wt % to about 7.0 wt %, about 0.10 wt % to about 8.0 wt %, about 0.1 wt % to about 9.0 wt %, and about 0.5% to about 7.5% of the present composition.

Additional embodiments of the present invention provide a novel co-mixture composition comprising: 1) a yeast cell wall extract(s), 2) a clay component(s), and 3) an algal material(s) admixed to 4) a proteinaceous and/or cell wall extract(s) from bacterial or fungal fermentation from amino acid production, in the presence or absence of one or more carrier(s). The addition of the algal material to the present composition and/or methods confirms that the adsorption efficacy toward a large set of mycotoxins could be maintained and was comparable and/or superior to the inventive compositions and methods that did not comprise the algal material.

In some embodiments of the present composition and methods comprising the algal material (“the algal embodiments”), the algal material is selected from a microalgae strain or a macroalgae strain. The microalgae strain or the macroalgae strain are produced under autotrophic, mixotrophic, or heterotrophic conditions. In some embodiments, the algal material is a microalgae produced under heterotrophic conditions.

In some embodiments the algal material is selected from an algal organism (i.e., an algae) from the divisions of Chlorophyceae (green algae), Chromophyta, Chriptophyta, Rhodophyta (red algae), Dinoflagellata (Pyrrophyta), Euglenophyta. In some embodiments, the algal material is selected from an organism in the classes of Chlorophyceae, Bacillariophyceae (diatoms), Chrysophyceae (golden algae), Xanthophyceae, Cryptophyceae, Euglininophyceae, Cyanophyceae, or combination thereof. In other embodiments, the algal material is from a heterotrophic strain of microalgae, such as Chlorella, Asterarcys quadricellulare, Aurantiochytrium, Schizochytrium, or other similar algal species. In additional embodiments, the algae or the algal material is Chlorella vulgaris.

In some embodiments, an effective amount of the algal material is from between about 1.0 wt % to about 99.0 wt % of the present composition, including any specific or variety of ranges of weight percent (wt %) comprised therein. In other embodiments, the effective amount of the algal material comprises more than about 99 wt % of the present composition. In further embodiments, the effective amount of the algal material is from between about 1.0 wt % to about 30.0 wt % of the present composition. In other embodiments, the effective amount of the algal material is from between about 1.0 wt % to about 10.0 wt % of the present composition. In further embodiments, the effective amount the algal material is from about 1.0 wt % to about 3.0 wt %, about 1.0 wt % to about 4.0 wt %, about 1.0 wt % to about 5.0 wt %, about 1.0 wt % to about 6.0 wt %, about 1.0 wt % to about 7.0 wt %, about 1.0 wt % to about 8.0 wt %, about 1.0 wt % to about 9.0 wt %, about 5.0 wt % to about 25 wt %, about 5.0 wt % to about 15 wt %, about 5.0 wt % to about 20 wt %, and about 5.0 wt % to about 10 wt % of the present composition.

In yet additional embodiments, the present composition may comprise no (0%) of the algal material. In further embodiments, the range of algal material in the present composition may range from about 0 wt % to about 30 wt %, from about 0 wt % to about 10 wt %, from 0.1 wt % to about 30 wt %, and from about 0.1 wt % to about 10 wt %.

The present disclosure is directed to one or more edible co-mixture compositions comprising: 1) a yeast cell wall extract(s), 2) a clay component(s), and 3) an optional algal material(s) admixed to 4) a proteinaceous and/or cell wall extract(s) from bacterial or fungal fermentation from amino acid production, in the presence or absence of one or more carrier(s). As such, the present composition may be applied as a feed ingredient, a feed additive, and/or an immobilized ingredient in animal feed blocks or boluses, but their use is not limited to these forms.

In some embodiments, the present compositions may be added, mixed, and/or combined, using any method known or employed in the animal feed production industry, with any animal feed product, component, and/or ingredient before, during, or after feed processing or production. In some embodiments, the present compositions and methods provided and/or comprised by any animal feed or feedstuffs, do not bind essential nutrients, such as vitamins and minerals, such that the nutritional value of the animal feed in which the present composition or methods are comprised is not decreased or reduced at all.

In this regard, the present disclosure is also related to one or more methods of producing and/or manufacturing the present composition. In some embodiments, the present method comprises admixing the effective amounts of yeast cell wall extract and the clay material, with or without the algae material, to a proteinaceous and/or cell wall extract(s) from bacterial or fungal fermentation from amino acid production. For example, some embodiments of the present method comprise drying and/or freeze drying the yeast cell wall extract, the proteinaceous and/or cell wall extract(s) from bacterial or fungal fermentation from amino acid production, and the algal material (if an algal embodiment) before mixing or admixing these components together. In some embodiments, the present method comprises admixing the clay material to the yeast cell wall extract prior to adding or mixing with the proteinaceous and/or cell wall extract(s) from bacterial or fungal fermentation from amino acid production.

Some embodiments of the present method comprise preparing, isolating, purifying, and/or concentrating the yeast cell wall extract. Previous method steps of preparing a yeast cell wall extract may include, cultivating a viable yeast cell, such as in a fermentation reactor or a petri dish. Some embodiment of the present method require further cultivating and/or isolating the yeast cell wall extract from the viable yeast cell in the presence or absence of a clay or a clay material or mixture. In some embodiments, the yeast cells are grown in the presence of and therefore comprise a clay and/or one or more clay component(s) that create a yeast-interlaced (e.g., via its yeast cell wall) clay structure. The yeast-interlaced clay structure may further comprise a glucan: mannan structure, which may or may not be altered (e.g., by the clay).

Some embodiments of the present method comprise blending or cultivating the yeast cell wall material with the clay, prior to admixing a yeast-clay mixture to the proteinaceous and/or cell wall extract(s) from bacterial or fungal fermentation from amino acid production. The present compositions and methods are not limited by any particular method of generating a yeast cell wall extract from yeast cells or the algal material from algal cells.

In further embodiments, the present method comprises admixing the clay to an algal material. The present method may also comprise obtaining the algal material from a heterotrophic or autotrophic fermentation process or fermenting the algal material in a heterotrophic or autotrophic process. For example, some embodiments of the present method comprise obtaining one or more microalgae cells from the algal material provided through heterotrophic or autotrophic growth.

Some embodiments of the present method of producing the present composition comprises collecting the proteinaceous and/or cell wall extract as a material (e.g., a waste material and/or a residual material) from a bacterial or fungal fermentation used for the production of amino acids. The method further comprises using or utilizing the proteinaceous and/or cell wall extract as a liquid extract or a dried or freeze-dried extract. In some embodiments, the cell wall extract from the bacterial fermentation used for the production of amino acids comprises a heteropolysaccharide meshwork cell wall composed of peptidoglycan, arabinogalactan-chained mycolic acids, glycolipids, S-layer glycoproteins, and/or combinations thereof, which are further extracted from the bacterial biomass rich in protein.

In some embodiments, the present method provides for the blending of one or more bacteria or fungal species (belonging to the genus described above, respectively) used for amino acid production or their extracted parietal components. In some method embodiments, the blending of the bacterial and/or fungal cell wall extract is composed of parietal individual components or combinations thereof comprising peptidoglycan; arabinan, galactan, chitin, mannan, glucan and combinations thereof; mycolic acids, including but not limited to, trehalose monomycolate, trehalose dimycolate, diacyltrehalose, polyacyltrehalose, phthiocerol dimycocerosate; glycolipids such as, but not limited to, sulfoglycolipid, phosphatidyl-myo-inositol mannosides, lipomannan, lipoarabinomannan, mannosylated lipoarabinomannan, S-layer glycoproteins, and/or combinations thereof.

In some embodiments, the present method provides for a blending or admixing of the clay or clay-based materials to the other components of the present compositions (e.g., the yeast cell wall extract and the proteinaceous and/or cell wall extracts). The blending or admixing process of the clay material comprises, consists of, but is not limited to, a dry blending method. For example, the dry blending method comprises of a weight-by-weight proportion of a dried yeast cell wall extract, a clay, an optional algal material, and a dried proteinaceous extract from bacterial or fungal fermentation from amino acid production or the extracted bacterial cell wall.

In some method embodiments, the amount of one or more proteinaceous and/or cell wall extract(s) from bacterial or fungal fermentation from amino acid production blended into the yeast cell wall extract and clay material, with or without addition of algal material, are present at a concentration of between about 0.1 wt % to about 99 wt %, including any specific or variety of ranges of weight percentages (wt %) comprised therein. For example, the amount of one or more proteinaceous and/or cell wall extract(s) from bacterial or fungal fermentation from amino acid production in the present composition is a concentration of less than about 0.5%, about 0.5-1%, 1-2%, 2-5%, 5-10%, 10-15%, 10-30%, 15-20%, 20-25%, 20-55%, 25-30%, 30-50%, 30-60%, 50-80%, 80-99% or more (on a w/w % basis) in a final dried product. The present composition also provides yeast cell wall extracts and clay, with or without addition of algal material, admixed to said proteinaceous material comprising an enhanced total protein content (e.g., 100%, 200%, 300%, 400%, 500% or more) compared to the total protein content of a yeast cell wall extracts and clay material alone, with or without addition of algal material.

In some embodiments, the amount of one or more clays blended into a yeast cell wall extract, with or without addition of algal material, are present at a concentration of between about 0.1 wt % to about 70 wt %, including any specific or variety of ranges of weight percentages (wt %) comprised therein. For example, the amount of one or more clays in the present composition are present at less than about 0.5%, about 0.5-1%, 1-2%, 2-5%, 5-10%, 5-20%, 5-25%, 10-15%, 15-20%, 20-25%, 25-30%, 30-50%, 50-70%, 70% or more (on a w/w % basis) of the final product content. In some embodiments, the amount of one or more clays blended into a yeast cell wall extract does not exceed 2% of the final content of the dried product. In some embodiments, the final concentration of clay blended with the composition comprises an amount of clay that has regulatory approval for use as a feed additive in non-medicated animal feed (e.g., does not exceed 2% in final product).

In some embodiments, the amount of one or more algal materials blended into the yeast cell wall extract and clay material, are present at a concentration of between about 0.1 wt % to about 99 wt %, including any specific or variety of ranges of weight percentages (wt %) comprised therein. For example, the amount of one or more algal materials in the present composition are present at a concentration of less than about 0.5%, about 0.5-1%, 1-2%, 2-5%, 5-10%, 10-15%, 15-20%, 20-25%, 25-30%, 30-50%, 50-80, 80-99% or more (on a w/w % basis) in the final dried product. In some embodiments, the algal material represents about 0% and is not present with the yeast cell wall extract, clay material and the proteinaceous and/or cell wall extract(s) from bacterial or fungal fermentation from amino acid production or the extracted bacterial cell wall at all.

In some embodiments, the amount of a carrier blended to a yeast cell wall extract and clay material, with or without addition of algal material, admixed to a proteinaceous and/or cell wall extract(s) from bacterial or fungal fermentation from amino acid production is present at a concentration of between about 0 wt % to about 50 wt % or about 1 wt % to about 50 wt %, including any specific or variety of ranges of weight percentages (wt %) comprised therein. For example, the amount of the carrier in the present composition is present at a concentration of about 0%, about 0.1-1%, 1-10%, 10-20%, 20-30%, 30-40%, 40-50% or more (on a w/w % basis) of the final product content.

Once the components of the present composition have been mixed or blended together, the composition is configured to be added to an animal feedstuff or a feed. In some embodiments, the present method comprises configuring the composition to be added, admixed, and/or adjoined to the feedstuff using any mechanism known in the animal feed or animal production arts. For example, the present composition may be added to a portion of an animal feed or an animal feedstuff in preparation for feeding and/or consumption by one or more animals. Additionally, the present composition may be admixed throughout the animal feed or the feedstuff prior to consumption by the animals.

Some method embodiments for producing the present composition comprise forming the composition in a dry, free-flowing powder suitable for direct inclusion into one or more animal feedstuffs, supplements, or other organic matter. In such embodiments, the animal feedstuff or animal feed of the present composition is selected from a Total Mixed Ration (TMR), a forage, a pellet, a concentrate, a premix, a coproduct, a grain, a distiller grain, a molasses, a fiber, a fodder, a grass, a hay, a kernel, a leaf or a plurality of leaves, a meal, one or more solubles, a supplement, and combinations thereof. In other embodiments, the animal feed or feedstuff in the present composition may be any organic or edible material fed to animals for any nutritional and/or medicinal purposes.

In some method embodiments, the present composition is configured to be formed into a pellet. As such, the present method of producing or manufacturing the composition further comprises pelleting the composition with the animal feedstuffs to form one or more animal feed pellets. One or more pellets, refers but is not limited to, one or more animal feed pellets that may or may not comprise the present composition. One embodiment of the present disclosure is an animal feed pellet that comprises the present composition for the subsequent nutritional consumption and/or medical treatment of animals by way of consumption and ingestion of the present composition to reduce presence, concentration, and/or harmful effects of mycotoxins in the animal (e.g., the gut of the animal).

The animal feed pellets may comprise a single feed or a mixed feed material, crumbled pellets, floating pellets, sinking pellets, cattle feed pellets, concentrated feed pellets, premix feed pellets, feed blocks, mineral blocks, and/or low moisture block supplements. Some embodiments of the present method of producing the present composition comprise utilizing and/or admixing the present composition into a textured feed or a sweet feed, extruding the feed, and then possibly granulating and/or prilling the animal feed or feedstuffs before feeding the.

In some embodiments, the present composition represents about 0.0125 wt % to about 10 wt % of an animal feedstuff, including any specific or variety of ranges of weight percentages (wt %) comprised therein. In some embodiments, for example, the effective amount of the present composition represents between about 0.0125 wt % to about 4.0 wt % of the animal feedstuff. In other embodiments, the effective amount of the present composition represents between about 0.05 wt % to about 2.0 wt % of the feedstuff. In further embodiments, the effective amount of the present composition represents from about 0.0125 wt % to about 3.0 wt %, about 0.0125 wt % to about 4.0 wt %, about 0.0125 wt % to about 5.0 wt %, about 0.0125 wt % to about 6.0 wt %, about 0.0125 wt % to about 7.0 wt %, about 0.0125 wt % to about 8.0 wt %, about 0.0125 wt % to about 9.0 wt %, about 0.05 wt % to about 5 wt %, about 0.05 wt % to about 10 wt %, about 0.05 wt % to about 8 wt %, and about 0.05 wt % to about 3 wt % of the present composition.

The present disclosure is further directed to a method of administering the present composition to an animal or an organic material. Moreover, the present method of administering the present composition to an animal further comprises a method of sequestering and/or adsorbing mycotoxins in an animal. Some of the present methods comprise co-administering the present composition to an animal via an animal feed or feedstuff in an amount effective to sequester and/or adsorb mycotoxins.

For example, the present composition may be administered and/or co-administered to an animal when the composition is mixed with a water, a liquid, a feedstuff, and/or other components utilized as an animal feed supplement or ingredient of an animal feed (“feed”). The feed may then be consumed by animals via direct feeding or indirect consumption, such that the composition is digested and relocated to a gut of the animal. When in the gut of the animal, the present compositions work to increase sequestration and/or adsorption of mycotoxins, which thereby reduces the bioavailability, absorption, or uptake of mycotoxin in the bloodstream of the animal, thereby enhancing performance, improving health and fertility, reducing the damaging effects, and decreasing the incidence of mycotoxin disease, infection, and/or contamination in animals.

When incorporated directly into an animal feed (“feed”) or an animal feedstuff, a present composition is added in amounts ranging from about 0.0125% to about 10% or from about 0.0125% to about 0.4% by weight of feed, including any specific or variety of ranges of weight percentages (wt %) comprised therein. In other embodiments, the present composition is added to the animal feedstuff at an inclusion rate of 0.125 to 4.0 kg/T of the feed, including any specific or variety of ranges of inclusion rates comprised therein. In some embodiments, a composition of the invention is added to feedstuffs in amounts from about 0.025% to about 0.2% by weight of the feedstuff. Alternatively, the present compositions may be directly fed to animals as a supplement in an amount ranging from about 2 to about 50 grams (g) per animal per day (2-50 g/animal/day), including any specific or variety of ranges of amounts comprised therein. In other embodiments, the present compositions are fed to animals as a supplement in an amount ranging from about 10 to about 30 grams (g) per animal per day (10-30 g/animal/day).

In some embodiments, the present composition is formulated to sequester and/or adsorb mycotoxins by co-administration of the composition for feeding, consumption, and/or contact by any animal member of Kingdom Animalia. In some embodiments, the animal member of Kingdom Animalia is selected from an avian, a bovine, a porcine, an equine, an ovine, a caprine, a piscine, a shellfish, a camelids, a feline, a canine, and a rodent species. In other embodiments, an animal member of Kingdom Animalia is a cow, a bull, one or more cattle, a pig, a swine, a sow, a chicken, a broiler, and/or a human.

Alternatively, in certain embodiments, the present compositions may be introduced to an animal via materials that the animals and/or humans may come into contact with. For example, the present composition comprising the yeast cell wall extract and clay material, with or without algae material, is admixed to a proteinaceous and/or cell wall extract(s) from bacterial or fungal fermentation from amino acid production may be configured to be adjoined and/or affixed to the animal feedstuff and/or an animal material component, such that that the composition may be located and/or positioned to sequester and/or adsorb mycotoxins that are present on (e.g., the skin or exterior surfaces of an animal) or within the animal (e.g., the gut, digestive system/tract, and/or cells of the animal whether located in vivo or in vitro). Specifically, the present composition may be admixed and/or comprised by an organic material and/or other materials that come into contact with animals (e.g., clothing or bedding), usage during food and beverage processing and manufacture, and usage during filtration of liquids.

For example, in some embodiments, the yeast cell wall extract and clay material, with or without algae material, is admixed to a proteinaceous extract from bacterial or fungal fermentation from amino acid production and is added or affixed to an organic material. When incorporated into other organic matter (e.g., animal bedding or clothing), the present composition may be added in amounts ranging from about 0.0125 wt % to about 99.9 wt % of the organic matter, including any specific or variety of ranges of weight percentages (wt %) comprised therein. In this way, interaction between the present composition and the animal will decrease the physical contact, uptake, penetration, bioavailability or absorption of mycotoxins in the bloodstream of the animal.

Alternatively, in some embodiments, the present composition is added to water or a liquid used for animal consumption, cleaning, and/or filtration purposes. For example, in some embodiments, a liquid is filtered through the yeast cell wall extract and clay material, with or without algae material, and admixed to a proteinaceous and/or cell wall extract(s) from bacterial or fungal fermentation from amino acid production. In those such embodiments, the liquid may be a juice, a water (e.g., distilled, sterile, and/or spring water), a beer, a wine, combinations thereof, and/or any other liquid that may serve a similar purpose. When incorporated into a liquid (e.g., water for filtration or other purposes), the present composition is added to the liquid in amounts ranging from about 0.0125 wt % to about 100 wt % of the organic matter including any specific or variety of ranges of weight percentages (wt %) comprised therein.

In some embodiments, the present composition and methods comprising the yeast cell wall extract, the clay material, with or without the algal material, and admixed to a proteinaceous and/or cell wall extract(s) from bacterial or fungal fermentation from amino acid production sequestered one or more mycotoxins. In some embodiments, the yeast cell wall extract and clay material admixed to a proteinaceous and/or cell wall extract(s) from bacterial or fungal fermentation from amino acid production (i.e., without the algal material) sequestered one or more mycotoxins. In some embodiments, the yeast cell wall extract, the clay, and the algal material admixed to a proteinaceous and/or cell wall extract(s) from bacterial or fungal fermentation from amino acid production sequestered one or more mycotoxins.

The present invention, composition, and/or method is not limited to the type of mycotoxin sequestered. Instead, the present composition and/or methods may be utilized to adsorb and/or sequester a variety of mycotoxins. Mycotoxins sequestered by the present compositions and methods are aflatoxins, ochratoxins, fumonisins, emerging Fusarium mycotoxins, Aspergillus mycotoxins, Penicillium mycotoxins, zearalenone, ergot alkaloids mycotoxins, consisting but not limited to AAL toxins, acetoxyscirpenediol, acetyldeoxynivalenol, acetylneosolaniol, acetyl T-2 toxin, acetyl HT-2, aflatoxins including aflatoxin B1 and B2 and G1 and G2, aflatoxicol, aflatrem, altenuic acid, alternariol, altertoxin, altersolanols, Alternaria toxins, apicidins, arugosins, asperazines, aspergillic acid, aspergillumarins, asperlicins, aspewentins, aspochalasins, aurofusarin, aurosperones, aurovertins, austalides, austdiol, austamide, austocystin, avenacein, baccharinoids, beauvericin, bentenolide, brevianamide, butenolide, calonectrin, chaetoglobosin, chevalones, citrinin, citreoviridin, citreoviridinol, cochliodinol, coniochaetons, cytochalasins, cyclosporins, cytochalasins, cyclopiazonic acid, deacetylcalonectrin, decarestrictine, deoxynivalenol, diacetoxyscirpenol, diacetyldeoxynivalenol, destruxins A and B, elymoclavines, enniatins such as enniatins A/A1 and B/B1, ergot toxins and endophytes such as ergine, ergocornine, ergocristine, ergocryptine, ergometrine, ergonine, ergosine, ergotamine, ergovaline, lysergol, lysergic acid, methylergonovine, and related epimers, fructigenines, fumigaclavines, fumagillin, fumiquinazolines, fumitremorgins, fumonisins including fumonisin A1 and B1 and B2 and B3, fusarenon X, fusaric acid, fusarin, fusarielin, fuscofusarin, geodin, geomycins, gliotoxin, griseophenones, griseofulvin, HT-2 toxin, ipomeanine, islanditoxin, isofumigaclavines A and B lateritin, leporisines, lolitrems, lycomarasmine, malformins, marcfortines, maleagrins, maltoryzine, miophytocens, moniliformin, monoacetoxyscirpenol, mycophenolic acid, neosolaniol, nigerapyrones, nivalenol, nordeodeoxynivalenol, NT-1 toxin, NT-2 toxin, ochratoxins such as ochratoxins A and B, oxalic acid, paraherquamide, paspalines, paspalitrems A and B, patulin, paxilline, penicillenol, penicillic acid, penitrems such as penitrem A, phomopsins, PR-toxin, psychrophilins, pyripyropenes, roridins, roritoxins, roquefortines such as roquefortine C, rubratoxin, rubroskyrin, rubrosulphin, rugulosin, satratoxins, scirpentriol, slaframine, solaniol, sporotrichiol, stephacisins, sterigmatocystin, sulochrin, swainsonine, T-2 toxin, tentoxin, terreins, territrems, tetrahydroaltersolanols, triacetoxyscirpendiol, trichothecenes, trichodermin, trichothecin, trichoverrins, trichoverrols, tryptoquivalene, verrucarins, versicolorins, versiconols, verruculogen, viopurpurin, viomellein, viriditoxin, wortmannin, xanthocillin, xanthomegnin, yavanicin, zanones, zearalenols, zearalanones, zearalenone and subfamilies, and/or possible conjugates and metabolites of the aforementioned mycotoxins, and/or combinations thereof. In some embodiments, the mycotoxins sequestered by the present compositions and methods are deoxynivalenol (DON), pencillic acid, fusaric acid, and/or combinations thereof.

Accordingly, the present compositions and methods lead to improved performance, better health and fertility, and a reduced incidence of disease, infection, and/or contamination in animals by limiting mycotoxin absorption or uptake in the bloodstream. Additionally, when these compositions are added to water intended for animal or human consumption or other uses, they similarly reduce mycotoxin bioavailability. Furthermore, the present compositions, can be utilized in liquid filtration processes effectively removing mycotoxins from food or liquids used for direct consumption by humans or animals or used in industrial processes, such as are often utilized in the food and/or beverage industry.

In some embodiments, co-administration of the present composition comprising the yeast cell wall extract, clay material, with or without algae material, and proteinaceous and/or cell wall extract(s) from bacterial or fungal fermentation from amino acid production sequestered a higher amount of mycotoxins than the composition comprising the yeast cell wall extract and clay material alone, with or without the algae material. In other words, the composition comprising the proteinaceous and/or cell wall extract(s) from bacterial or fungal fermentation from amino acid production sequestered more mycotoxins than the composition that did not have that component. Thus, it is clear that the addition of the proteinaceous and/or cell wall extract(s) from bacterial or fungal fermentation from amino acid production component provides unexpected technical and beneficial effects on the present composition and its ability to provide improved mycotoxin sequestration in animals over other compositions not comprising that component.

In some embodiments, co-administration of the present composition comprising the yeast cell wall extract and clay material, with or without the algae material, and proteinaceous and/or cell wall extract(s) from bacterial or fungal fermentation from amino acid production sequestered a higher amount of mycotoxins than the proteinaceous extract from bacterial or fungal fermentation from amino acid production alone. In other words, the composition comprising the yeast cell wall extract, clay material, and the proteinaceous extract from bacterial or fungal fermentation from amino acid production sequestered more mycotoxins than the composition that did not have all of those components.

The novel, edible, co-mixed, and/or co-administered compositions described herein have shown synergistic properties in adsorption, sorption, sequestration, and binding of mycotoxins inside the digestive tract of animals. The addition of the algal material to the yeast cell wall, the clay material, and/or the proteinaceous and/or cell wall extract(s) from bacterial or fungal fermentation from amino acid production showed an even further improvement and enhancement of some binding features to mycotoxins that were also demonstrated (to a lesser degree) by the composition without the algal material. Therefore, the algal embodiment of the present composition further extends the capability and synergistic activity and effect previously observed with the present composition embodiment comprising the yeast cell wall extract(s) and the clay material(s) admixed to a proteinaceous extract(s) from bacterial or fungal fermentation from amino acid production (i.e., no algal material). Accordingly, the addition of the algal material component provides unexpected technical and beneficial effects on the present composition and its ability to provide improved mycotoxin sequestration and/or adsorption, particularly in animals.

Establishment of the efficacy of the present composition comprising the yeast cell wall, the clay material, and the proteinaceous and/or cell wall extract(s) from bacterial or fungal fermentation from amino acid production was further investigated in vivo through administration of a co-mixture to one or more broiler chicken(s). In addition, mycotoxin distribution in animal blood was also evaluated in the presence or absence of the present composition. The present novel composition embodiments have shown a positive impact on the digestive volatile fatty acid distribution and concentrations in vitro and/or in vivo, as well as the microbial phyllosphere population within the animal gut dynamic favoring the presence of beneficial microbes over pathogenic ones.

The present composition embodiment comprising the algal material is based on the present composition without the algal material that introduced a novel combination of yeast cell wall extract and clay material mixed with a proteinaceous and/or cell wall extract(s) derived from bacterial or fungal fermentation in amino acid production. The present compositions, or those derived therefrom, along with their methods of use (such as sequestering/adsorbing mycotoxins or destabilizing mycotoxins), offer new approaches for mycotoxin mitigation and reduction of mycotoxin contamination and/or infection, and especially highlight a synergistic and beneficial effect between the multiple components of the present composition (i.e., the yeast cell wall extract, the clay, and the proteinaceous and/or cell wall extract derived from bacterial or fungal fermentation in amino acid production, with or without the algal material) that could not and has not previously been identified and/or demonstrated from their individual ingredient efficacy evaluation or their theoretical or predicted additive efficacy. Thus, their combination in the present composition provides synergistic effects with respect to reducing, preventing, destabilizing, and/or mitigating mycotoxin contamination and/or infection of animals and/or humans.

Another aspect of the present disclosure is to further establish the mitigation properties of the present composition comprising the yeast cell wall extract and clay material mixed with a proteinaceous and/or cell wall extract(s) derived from bacterial or fungal fermentation in amino acid production. Experiments were conducted in vivo with animals receiving the present composition in the presence of a natural mycotoxin challenge. The influence of this mitigation in situ was also evaluated in a third step on the digestive microbiota collected from the caeca of animals in a poultry trial with identified biomarkers, showing changes in fermentation capabilities, as well as beneficial shifts in microbial populations. This evidence demonstrates the potential mitigating impact the present compositions and methods can have on mycotoxin pathogen interference, prevention or reduction of mycotoxin infection, disease, and/or contamination, and reduction or mitigation of the numerous overall harmful effects in the animals due to mycotoxin contamination and/or infection.

In some embodiments, the present invention provides a novel co-mixture composition comprising a yeast cell wall extract and clay, with or without addition of algal material, admixed to the said bacterial biomass rich in protein material in altering the protein composition of the final composition and when admixed or co-administered to feedstuffs, water, and/or other organic materials, is able to sequester mycotoxins (e.g., while in the digestive system of an animal that consume said composition), and mitigating or reducing the negative effects of mycotoxins to the animal. In some embodiments, the present invention provides methods that significantly improve the adsorptive/sequestering properties of yeast cell wall-based material toward mycotoxins.

Accordingly, the present disclosure is directed to a method of adsorbing and/or sequestering mycotoxins. For example, the present compositions improve the adsorption of mycotoxins, such as deoxynivalenol (DON), penicillic acid, and/or fusaric acid, improve the adsorption of a wider variety of mycotoxins, such as mycotoxins that are not adsorbed and/or sequestered using components of the present composition individually (e.g., the yeast cell wall extract alone, the clay alone, the bacterial biomass rich in protein alone, and/or combinations of the yeast cell wall extract and clay material only).

In some embodiments, the present composition is provided in the co-administration of yeast cell wall extract and a clay, with or without addition of algal material, with a proteinaceous and/or cell wall extract(s) from bacterial or fungal fermentation from amino acid production that provides superior mycotoxin sequestration properties than conventional compositions. In some embodiments, the present invention combining yeast cell wall extract and clay, with or without addition of algal material, with proteinaceous and/or cell wall extract(s) from bacterial or fungal fermentation from amino acid production provides superior mycotoxin sequestration properties than each individual components including yeast cell wall extract or clay material or algal material or proteinaceous extract from bacterial or fungal fermentation from amino acid production alone.

Experiments conducted during the development of the present composition demonstrate the benefit(s) of using the present compositions and/or methods compared to historical or conventional methods. For example, as described herein, a drawback of using a traditional composition comprising only a (1) yeast cell wall extract and clay material alone or in combination or (2) a composition comprising a proteinaceous extract from bacterial or fungal fermentation from amino acid production, is that such compositions and methods of using the same are a less effective means than the present composition comprising all of those components, with or without addition of algal material, and specifically for reducing the negative effects of mycotoxins and have more drawbacks. The use of the algal material in the present invention only further increases the benefits observed for the use of the present composition comprising or consisting essentially of a yeast cell wall extract and clay material admixed to a proteinaceous and/or cell wall extract(s) from bacterial or fungal fermentation from amino acid production.

In some embodiments, the present invention also displays unexpected technical results in its ability to sequester a variety of a number or a plurality of mycotoxins (e.g., 3 or more, about 1-10, 8-15, 1-1000, 5-500, 10-250, 20-100, 100-500, 500-1000, greater than 1000, etc.) that have not been investigated or reported into the scientific literature previously, such as emerging mycotoxins. The present compositions and methods also display a strikingly improved or enhanced ability to adsorb mycotoxins compared to conventional compositions (e.g., a composition comprising only clay(s), a composition comprising only a yeast cell wall extract, a composition comprising a yeast cell wall extract and a clay(s) material only, a composition only comprising a proteinaceous and/or cell wall extract(s) from bacterial or fungal fermentation from amino acid production, and/or a composition comprising a yeast cell wall extract, a clay(s) material and an algal material only, and/or other combinations that do not comprise all three of the components, with or without the algal material).

In some embodiments, the present composition is able to adsorb between from about 33% to about 83% of deoxynivalenol (DON), including any specific or variety of ranges of adsorption percentage comprised therein (e.g., from about 75.5% to about 83% DON), with an average adsorption of 60.9%. Historical, traditional, and/or conventional methods using yeast cell wall and clay material, with or without addition of algal material, were only able to adsorb less than 0.5% to about 75.3% of deoxynivalenol (DON) with an average adsorption of 28.8%. Thus, the present compositions demonstrate an ability to have a greater adsorption range and wider capacity to bind and remove well known mycotoxins, such as deoxynivalenol (DON).

In addition, the present composition is able to adsorb between from about 33% to about 100% of fusaric acid, including any specific or variety of ranges of adsorption percentage comprised therein (e.g., from about 81% to about 100% fusaric acid), with an average adsorption of 76.3%. Historical, traditional, and/or conventional methods using yeast cell wall and clay material, with or without addition of algal material were only able to adsorb from about less than 0.5% to about 80.9% of fusaric acid with an average adsorption of 26.8%. Thus, the present compositions demonstrate an ability to have a greater adsorption range and wider capacity to bind and remove well known mycotoxins, such as fusaric acid.

Notably, the present composition is also able to simultaneously adsorb between from about 60% to about 75% of deoxynivalenol (DON) and between from about 76% to about 97% of fusaric acid. Thus, the present compositions demonstrate an ability to have a greater adsorption range and wider capacity to simultaneously bind and remove well known mycotoxins, such as deoxynivalenol (DON) and fusaric acid.

Accordingly, the present compositions and methods presented herein consisting of yeast cell wall extract and a clay, admixed with a proteinaceous and/or cell wall extract(s) from bacterial or fungal fermentation from amino acid production represents an improvement in mycotoxin mitigation efficacy of about 211% for deoxynivalenol (DON) adsorption and/or sequestration over historical, traditional, or conventional methods using yeast cell wall and clay material. In addition, the present compositions and methods represent an improvement in mycotoxin mitigation efficacy of 285% for fusaric acid sequestration and/or adsorption over historical, traditional, or conventional methods using yeast cell wall and clay material.

Further, the present methods and compositions maintained high adsorption capacity toward aflatoxins, with average adsorption ranging between from about 78% to about 100%; toward ochratoxins from about 55% to about 66% average adsorption; toward trichothecenes from about 33 to 93% average adsorption; toward emerging fusarium mycotoxins from about 39% to 100% average adsorption; toward fumonisins from about 30% to about 58%; for zearalenone from about 43% to about 88%; for Penicillium toxins from about 28% to about 79% average adsorption; for Aspergillus toxins from about 79% to about 100% average adsorption; and for ergot alkaloids from about 45% to about 87% average, including any specific or variety of ranges of adsorption percentage comprises therein. These results demonstrated the improved efficacy of the present compositions to bind a greater variety of mycotoxins at a higher average adsorption efficacy rate than traditional and/or historical compositions.

The addition of the algal material to the present composition comprising the yeast cell wall extract and clay material admixed to a proteinaceous and/or cell wall extract(s) from bacterial or fungal fermentation from amino acid production composition provided a significant increase of the adsorption for citrinin, with 88% of the toxin bound, 74% of penitrem A being sequestered, and 46% of alteroxin-I being bound. The present composition of yeast cell wall extract and a clay, with or without addition of algal material, with herein proteinaceous bacterial extract showed an improvement in adsorption capabilities for deoxynivalenol from 161% to 259% increase in sorption ability, respectively. Similarly, the present invention compositions comprising the algal material significantly improved the sequestration capabilities toward fusaric acid with about 72% and 79% toxin bound, respectively, accounting for an increase of 124% to 168%, respectively. The present invention compositions also had beneficial features over the sequestration of ochratoxin A, T-2 toxin, aurofusarin, mycophenolic acid, alternariol, tentoxin and provided similar high binding efficacy toward, but not limited to, aflatoxins, beauvericin, enniatins, ochratoxin A, penicillic acid, paxillin, alternariol, cyclopiazonic acid and in a lesser extent chaetocin, fumagillin, sporidesmin, fumitremorgin C.

The present compositions, or those derived therefrom, along with their methods of use (e.g., sequestering mycotoxins), offer new approaches for mycotoxin mitigation and especially highlights an observed or identified synergy between the present components that could not be seen from individual feed ingredient efficacy evaluation and/or their theoretical additive efficacy prediction. Another aspect of the present invention is to further establish the mitigation properties of the yeast cell wall extract and clay material mixed with a proteinaceous and/or cell wall extract(s) derived from bacterial or fungal fermentation in amino acid production, in vivo with an animal receiving the product in the presence of a natural mycotoxin challenge. As demonstrated herein and described in Examples 1 to 8 below, the compositions of the present invention provide a significant and unexpected enhanced and/or improved technical ability to adsorb, sequester, and/or bind mycotoxins, specifically in animals and/or other organic materials, as compared to other historical, traditional, and/or conventional compositions that did not comprise most or all of these components.

### EXPERIMENTAL DATA

The following examples are provided in order to demonstrate and further illustrate certain embodiments and aspects of the present compositions and methods and are not to be construed as limiting the scope thereof.

### Example 1. Parsing the In Vitro Efficacy of Sequestration of a Yeast Cell Wall Extract and Clay Material Admixed to a Proteinaceous Extract from Bacterial Fermentation from Amino Acid Production, the Proteinaceous Extract from Bacterial Fermentation from Amino Acid Production, Coarse Yeast Cell Wall and Clay Material Mixture, Yeast Cell Wall Extract and Clay Material Admixed to Algal Cells

One formulation embodiment of the present inventive composition (1) comprised the yeast cell wall extract and clay material admixed to a proteinaceous and/or cell wall extract from a bacterial or fungal fermentation from amino acid production was prepared. This specific embodiment comprised a bacterial protein biomass. As such, this original formulation embodiment of the present composition (1) comprised about 40-60 wt % of a yeast cell wall extract and about 5-20 wt % of a clay material, which was collectively admixed to about 20-55 wt % of a proteinaceous extract of a bacterial fermentation from amino acid production (i.e., bacterial protein biomass). The composition (1) was admixed together for further testing.

Four compositions were then tested, including composition (1) a yeast cell wall extract and clay material admixed to a proteinaceous extract from bacterial fermentation from amino acid production (e.g., prepared as previously described above) and composition (2) a proteinaceous extract from bacterial fermentation from amino acid production alone, were tested and compared to historical or traditional compositions, such as composition (3) a coarse yeast cell wall and clay material mixture, and composition (4) a yeast cell wall extract and clay material admixed to algal cells, each at the same weight to volume ratio, were tested in vitro for their efficiency at interacting with two different mixtures of mycotoxins in a citrate buffer environment of pH 3.0, as a condition of the proximal digestive tract in monogastric animals.

An amount of 0.1 g of samples (1), (2), (3) and/or (4) was weighed and placed in microcentrifuge tubes and a suspension of 1 ml of ammonium citrate buffer maintained at pH 3.0. A 24 uL slurry sample of each product was then used and applied to a 576 uL mixture of mycotoxins. Two mixtures of mycotoxins (e.g. Mycotoxin Mixtures A-D) were evaluated, comprising concentrations of mycotoxins generally reported and found in feedstuffs, and ranging from about 10 ng/ml to about 500 ng/ml (e.g., Mycotoxin Mixture A), from about 10 ng/ml to about 1500 ng/ml (e.g., Mycotoxin Mixture B), including an specific or variety of ranged of mycotoxin concentration comprised therein, including at or about 1000 ng/ml for each mycotoxin (e.g., Mycotoxin Mixtures C and D). The mycotoxin mixtures were placed in a Pierce Spin Column cassette equipped with a frit and filter disc to generate a final concentration of the adsorbent of 0.4% weight by volume. This process was repeated for each replicate and product.

The samples were then incubated 60 minutes on a rocker platform at a low speed, equivalent to 150 rpm at 37° C. After reaction, the cassettes were centrifuged at 10,000 rpm for 2 minutes. A volume of 200 μL of each sample was transferred to 1 mL silanized glass low volume recovery UPLC vial before being loaded into a UPLC-MS sampler and quantified. Mycotoxins 13C-internal standards were spiked to each sample to correct for signal suppression or enhancements during the detection process by means of mass spectrometry.

Mycotoxins were separated using a CORTECS reverse-phase C18 column (2.7×100 mm, 1.6 um, Waters Corp.), maintained at 40° C., and using a gradient of a dual mobile phase consisting in OptimaGrade water with 0.1% formic acid and methanol MS grade solvent with 0.1% formic acid, maintained at a flow rate of 0.3 mL/min by an Acquity UPLC system (Waters Corp.). A volume of 10 μL of sample was injected in the system. The UPLC system was interfaced with the IMS-QTOF-MSE mass spectrometer (Vion™, Waters Corp.), fitted with an electro-spray ionization (ESI) source, and working in positive and negative mode. The mass analyzer conditions and parameters were optimized according to common methods used by operators skilled in the art.

The results representing the percent of adsorption of every toxin investigated within 2 different multi-mycotoxin mixtures (i.e., Mycotoxin Mixtures A and B) are presented in Table 1. Mycotoxin Mixture A comprised each mycotoxin at a concentration ranging from about 10 ng/ml to about 500 ng/ml, as specified below. Mycotoxin Mixture B comprised each mycotoxin at a concentration ranging from about 10 ng/ml to about 1500 ng/ml, as specified below. The specific composition of the Mycotoxin Mixtures A and B are also described in Table 1, although the results represent many different mixtures of mycotoxins that were tested to demonstrate the same results.

Percent of adsorption was calculated by differential measurement of control samples comprising the mycotoxins only in buffer and the free mycotoxins present in the supernatant of samples treated with the compositions described herein. Concentrations were established from the integration of the area under the curve for the chromatograms generated after elution and separations of the investigated analytes by UPLC, and extraction of the total ion charts generated by mass spectrometry of each mass-to-charge values specific to each analytical target. Concentrations of each experimental samples were produced from the signal intensity detected by the mass spectrometer compared to control analytical standard curves of a minimum of 5 concentrations points, normalized by 13C-labelled mycotoxin internal standards.

Data presented in Table 1 suggests that based on the overall average adsorption, a dramatic increase of the sequestration efficacy of the inventive compositions was observed when (1) the yeast cell wall extract and clay material was admixed to a proteinaceous extract from bacterial fermentation from amino acid production over historical/traditional formulations (3) and (4). Specifically, the present composition had an average adsorption of about 67% for sample (1) compared to between from about 39% for sample (3) to about 44% for sample (4). In addition, the present composition of sample (1) composed of yeast cell wall extract and clay material admixed to a proteinaceous extract from bacterial fermentation from amino acid production had a higher sequestering activity over the composition (2) of proteinaceous extract from bacterial fermentation from amino acid production used alone, with an average adsorption of about 67% for the present composition (1) over about 50% for sample (2).

Sorption capabilities for the invention compositions (1) were for AFB1 about 100%; about 75% for Roquefortine C, Fumonisin B1 and ochratoxin A and zearalenone; between from about 43 to about 60% for deoxynivalenol and fusaric acid, respectively; and about 30% for T2-toxin. The results obtained showed an unsuspected behavior of the novel composition specifically toward deoxynivalenol and fumonisin B1 with the mixture of mycotoxins tested herein. For both toxins, the average adsorption of the present compositions was higher than just the additive adsorption obtained from the historical composition mainly composed of yeast cell wall extract (samples 3 and 4) and the proteinaceous material alone of sample (2). The proteinaceous material alone (2) showed, with about 28% and about 43% binding, an important contribution to the binding of deoxynivalenol or fumonisin B1 of the novel composition (1). The combination of the yeast cell wall extract and clay material to the proteinaceous extract from bacterial fermentation from amino acid production further improved by about 154% deoxynivalenol sequestration.

Overall, the results produced in this adsorption assay represented important breakthrough as compositions of the invention showed one of the highest binding adsorption ever recorded and significant improvements over historical or traditional compositions, and important attributes especially toward some of the historically most difficult toxin to interact with.

TABLE 1
In vitro multi-mycotoxins adsorption characteristics of (1) yeast cell wall extract
and clay material admixed to a proteinaceous extract from bacterial fermentation from
amino acid production, (2) the proteinaceous extract from bacterial fermentation from
amino acid production, (3) coarse yeast cell wall and clay material mixture, and (4)
yeast cell wall extract and clay material admixed to algal cells at pH 3.0 for two
mixtures of mycotoxins (Mycotoxin Mixtures A and B) commonly found in feedstuffs.

| Mycotoxin | | | | | |
|---|---|---|---|---|---|
| Mixtures A and | Initial | Percent Adsorption/Sequestration/Binding (%) | | | |
| B | (ng/mL) | Composition (1) | Composition (2) | Composition (3) | Composition (4) |
| Mycotoxin | 500 | 73.0 | 42.9 | 12.6 | 21.8 |
| Mixture A | | | | | |
| Fumonisin B1 | | | | | |
| Roquefortine C | 10 | 75.4 | 49.0 | 73.9 | 78.0 |
| Deoxynivalenol | 200 | 42.9 | 27.6 | 3.4 | 0.0 |
| T2-toxin | 20 | 27.8 | 31.3 | 23.0 | 27.3 |
| Aflatoxin B1 | 50 | 100.0 | 39.8 | 94.9 | 97.9 |
| Zearalenone | 20 | 76.2 | 76.6 | 38.9 | 47.6 |
| Ochratoxin A | 10 | 75.2 | 76.4 | 56.1 | 77.7 |
| Fusaric acid | 60 | 59.9 | 38.8 | 38.5 | 59.6 |
| Average Adsorption (%) | | 66.3 | 47.8 | 42.7 | 51.2 |
| Mycotoxin | 1500 | 62.4 | 23.7 | 2.8 | 6.4 |
| Mixture B | | | | | |
| Fumonisin B1 | | | | | |
| Roquefortine C | 10 | 74.2 | 45.2 | 66.3 | 71.3 |
| Deoxynivalenol | 500 | 48.2 | 41.8 | 0.0 | 0.0 |
| T2-toxin | 30 | 28.0 | 44.7 | 15.8 | 0.0 |
| Aflatoxin B1 | 50 | 100.0 | 39.0 | 89.8 | 95.9 |
| Zearalenone | 40 | 76.6 | 81.4 | 27.9 | 34.1 |
| Ochratoxin A | 50 | 86.6 | 96.6 | 49.5 | 62.1 |
| Fusaric acid | 120 | 66.5 | 49.8 | 38.8 | 19.9 |

| | | | | |
|---|---|---|---|---|
| Average Adsorption (%) | 67.8 | 52.8 | 36.4 | 37.8 |
| Total Average Adsorption (%) | 67.1 | 50.3 | 39.5 | 43.7 |

### Example 2. Comparing Different Subsets of the Present Compositions, with Different Inclusion Levels of a Proteinaceous Extract from Bacterial Fermentation from Amino Acid Production into Different Sources of Yeast Cell Wall Extracts

Four compositions of the present composition (1) comprising 2 sources of yeast cell wall extract and clay material ([A,B] and [D,E]) were admixed at 2 different inclusion rates of a proteinaceous extract from bacterial fermentation from amino acid production ([A,D] and [B,E]). Specifically, composition formulas A and B use the same amount of yeast cell wall (e.g., 50%), as do composition formulas D and E (e.g., 30%). However, composition formulas A and D use similar yeast cell wall preparations (e.g., prep 1), while composition formulas B and E also use similar yeast cell wall preparations (e.g., prep 2). Additionally, composition formulas A and B comprise the same inclusion rate of proteinaceous extract of bacterial fermentation (PEBF) at 35%, while composition formulas D and E comprise the same inclusion rate of proteinaceous extract of bacterial fermentation (PEBF) at 55%.

TABLE 2
Composition formulas A, B, D, and E were prepared similar to
the original formulation of composition (1), as described in
Example 1. These compositions formulas were prepared comprising:
1) about 25 wt % to about 50 wt % of yeast cell wall (e.g.,
extract), 2) about 5 wt % to about 25 wt % of clay material,
and about 30 wt % to about 60 wt % of proteinaceous extract
from bacterial fermentation from amino acid production. Notably,
composition formula J comprised about 30 wt % to about 60 wt
% of proteinaceous extract from bacterial and fungal fermentation
from amino acid production, which was utilized in the experiments
described in Example 4 below.

| Composition | Yeast cell | Clay | | |
|---|---|---|---|---|
| Formula | wall | material | PEBF* | PEFF** |
| A*** | 50% prep 1 | 15% | 35% | |
| B*** | 50% prep 2 | 15% | 35% | |
| D**** | 30% prep 1 | 15% | 55% | |
| E**** | 30% prep 2 | 15% | 55% | |
| J**** | 40% | 15% | 10% | 35% |

*Proteinaceous extract of bacterial fermentation (PEBF)
**Proteinaceous extract of fungal fermentation (PEFF)
***evaluating the effects of different cell wall preparations (e.g., prep 1 and prep 2)
****evaluating different composition formulas

The sample were tested, evaluated, and compared to the original formulation of the present composition ((1), C), as well as a proteinaceous extract from bacterial fermentation from amino acid production tested alone (2), and the historical formulation embodiment (4). The sample were specifically analyzed for their efficiency at sequestering, interacting, binding, and/or absorbing to two different mixtures of mycotoxins in a citrate buffer environment of pH 3.0, as a condition of the proximal digestive tract in monogastric animals.

The same procedure as in Example 1 was followed for the preparation and analysis of the different tested samples in this example. In some embodiments, the inclusion levels of proteinaceous extract from bacterial fermentation from amino acid production was between from around 10% to around 30%, including any specific or variety of ranges of inclusion levels comprised therein.

The results shown in Table 3 indicate that while the tested formulas share some common features, there are notable differences in their overall adsorption efficacy. The average mycotoxin percentage of sequestration for formulas A,C,D were about 60%, comparable to the previously tested composition (1). Compositions of formulation B and E, with average adsorption percentage from about 51% to about 53%, were less effective, demonstrating an impact of the proteinaceous bacterial extract inclusion level, which was different from A,C,D formulations. Composition only including proteinaceous extract from bacterial fermentation from amino acid production (2) showed improved performance over example 1, surpassing the 60% sequestration average percentage.

Individual mycotoxin interactions in Mycotoxin Mixtures A and B varied among tested compositions, and are ranked from best to worst as follows.
Aflatoxin B1: Formulas A through E were all effective in binding aflatoxin B1, consistent with previously tested composition (1). No free AFB1 was detectable in the media for formulas A to D. Formula (2) also demonstrated satisfactory binding to aflatoxin B1, although its efficacy was slightly below average compared to prior data. This suggests potential instability in aflatoxin B1 interaction with the proteinaceous extract from bacterial fermentation possibly due to non-specific mycotoxin-peptidic/protein interactions.
Ochratoxin A: All formulations interacted with at least about 69% of ochratoxin A, with formulas D and E reaching up to 83%, approaching the adsorption values observed with composition (1). The difference observed tended to acknowledge a difference between yeast cell wall extract sources but also showed an improved of the sorption percentage value with higher titers of the proteinaceous bacterial extract.
Roquefortine C: All formulas effectively bound over 50% of roquefortine C. Formulas A,D, (2) were particularly effective, showing between about 73% to about 75% of adsorption efficacy, whereas formulas B,E demonstrated interaction between from about 53% to about 58%. This showed a direct effect of the inclusion level of the proteinaceous bacterial extract on roquefortine C interaction.
Zearalenone: Formulas B,C,E showed the lowest adsorption efficacy toward zearalenone, about 50%, while formulas A,D, (2) reached up to 79% adsorption, showing both an effect from the proteinaceous bacterial extract inclusion level and the yeast cell wall extract origin.
Fusaric acid: Formula C demonstrated the best performance in binding fusaric acid, with efficacy between 67% and 77%. Only the historical formula (4) showed comparable performance.
Deoxynivalenol: Composition C achieved the highest adsorption percentage for deoxynivalenol, ranging from about 65% to about 68%. Other formulations showed an average of about 39% efficacy. The invention composition C (aka. (1)) showed a clear combinatorial effect toward deoxynivalenol mitigation, particularly since the inclusion of proteinaceous bacterial extract improved deoxynivalenol adsorption compared to historical formulations (4). The proteinaceous bacterial extract (2) seemed to be a clear contributor to the sequestration of deoxynivalenol, further augmenting the adsorption when present in combination with the yeast cell wall extract.
Fumonisin B1: Formulas A and, to a lesser extent, D exhibited the highest relative efficacy towards fumonisin B1. Composition (2) alone demonstrated the highest adsorption performances, ranging from 50% to 73%. Fumonisin B1 adsorption varied significantly among products, with an average of 40±15%. An antagonistic effect of the addition of the yeast cell wall toward fumonisin B1 was observed, the proteinaceous bacterial extract inclusion affecting positively the efficacy.
T-2 Toxin: None of the formulas exhibited adsorption over about 22% for T-2 toxins. The proteinaceous bacterial extract alone had comparable effect to the yeast cell wall extract.

Overall, these results underscore the effectiveness of composition (1) and its formulation C and A as particularly effective for targeting deoxynivalenol and fumonisin B1, respectively, more effectively than previously tested formulations. Composition (2) showed improved affinity for fumonisin B1.

TABLE 3
In vitro multi-mycotoxins adsorption characteristics of combinations of 2 sources of
yeast cell wall extract and clay material ([A, B] and [D, E]) admixed to
2 different inclusion rates of proteinaceous extract from bacterial fermentation from
amino acid production ([A, D] and [B, E]) and (2) a proteinaceous extract
from bacterial fermentation from amino acid production alone (2) compared to the original
formulation of composition ((1) i.e., C) and historical formulation embodiment (4)
at pH 3.0 for two mixtures of mycotoxins commonly found in feedstuffs.

| Mycotoxin | Percent Adsorption/Sequestration/Binding (%) | | | | | | | |
|---|---|---|---|---|---|---|---|---|
| Mixtures A | Initial | | | Comp C | | | | |
| and B | (ng/ml) | Comp A | Comp B | (i.e., (1)) | Comp D | Comp E | Comp (2) | Comp (4) |
| Mycotoxin | 500 | 44.2 | 22.4 | 19.9 | 50.9 | 23.2 | 64.2 | 21.8 |
| Mixture A | | | | | | | | |
| Fumonisin B1 | | | | | | | | |
| Roquefortine C | 10 | 67.3 | 55.0 | 57.1 | 68.6 | 56.2 | 72.2 | 78.0 |
| Deoxynivalenol | 200 | 33.5 | 39.3 | 67.9 | 43.9 | 40.6 | 48.7 | 0.0 |
| T2-toxin | 20 | 0.0 | 10.8 | 17.0 | 17.2 | 16.4 | 14.5 | 27.3 |
| Aflatoxin B1 | 50 | 100.0 | 100.0 | 100.0 | 100.0 | 100.0 | 99.9 | 97.9 |
| Zearalenone | 20 | 53.1 | 47.0 | 48.4 | 64.1 | 57.2 | 64.0 | 47.6 |
| Ochratoxin A | 10 | 71.9 | 68.4 | 72.6 | 78.4 | 79.3 | 77.2 | 77.7 |
| Fusaric acid | 60 | 49.6 | 48.5 | 66.7 | 53.5 | 49.6 | 50.7 | 59.6 |
| Average Adsorption (%) | | 52.1 | 48.9 | 56.2 | 59.6 | 52.8 | 61.4 | 51.2 |
| Mycotoxin | 1500 | 64.8 | 31.1 | 29.0 | 50.6 | 31.8 | 70.7 | 6.4 |
| Mixture B | | | | | | | | |
| Fumonisin B1 | | | | | | | | |
| Roquefortine C | 10 | 74.6 | 53.0 | 62.3 | 67.9 | 58.2 | 72.9 | 71.3 |
| Deoxynivalenol | 500 | 43.8 | 37.5 | 64.7 | 38.9 | 35.1 | 44.6 | 0.0 |
| T2-toxin | 30 | 14.4 | 15.2 | 21.5 | 18.3 | 16.4 | 20.0 | 0.0 |
| Aflatoxin B1 | 50 | 100.0 | 100.0 | 100.0 | 100.0 | 84.7 | 100.0 | 95.9 |
| Zearalenone | 40 | 70.8 | 59.4 | 61.1 | 71.1 | 65.6 | 70.7 | 34.1 |
| Ochratoxin A | 50 | 81.3 | 74.8 | 77.9 | 82.9 | 78.6 | 80.6 | 62.1 |
| Fusaric acid | 120 | 64.1 | 51.9 | 77.1 | 55.8 | 54.4 | 54.3 | 19.9 |

| | | | | | | | |
|---|---|---|---|---|---|---|---|
| Average Adsorption (%) | 64.2 | 52.9 | 61.7 | 60.7 | 53.1 | 64.2 | 37.9 |
| Total Average Adsorption | 58.2 | 50.9 | 58.9 | 60.1 | 53.0 | 62.8 | 43.7 |
| (%) | | | | | | | |

### Example 3. Effect of Heat Deactivation on the Activity of Previously Tested Formulations Investigated in Example 2

Thermal inactivation of the five compositions previously tested and described in Example 2, of 2 sources of yeast cell wall extract and clay material ([A,B] and [D,E]) admixed to 2 different inclusion rates of proteinaceous extract from bacterial fermentation from amino acid production ([A,D] and [B,E]) and (2) a proteinaceous extract from bacterial fermentation from amino acid production tested alone were evaluated and compared to the original formulation ((1) aka., C) and historical formulation embodiment (4) for their efficiency at interacting with two different mixtures of mycotoxins (Mycotoxin Mixtures A and B) in a citrate buffer environment of pH 3.0, as condition of the proximal digestive tract in monogastric animals. In addition, the evaluation of a non-deactivated and propagated bacteria from Corynebacterium genus (5) was evaluated for its detoxification capacity.

The same procedures as in Examples 1 and 2 were followed for the analysis of the preparation and analysis of the different tested samples. In some embodiments, the inclusion levels of proteinaceous extract from bacterial fermentation from amino acid production was between 10 and 30%. Inactivation of the five different compositions was performed using an autoclave set up at a temperature of 121° C. for 40 minutes.

After deactivation of the previously tested compositions A,C,D, and E, all previous observations and conclusions held true demonstrating that the effect observed is associated with a physicochemical interaction rather than any biological active detoxification process (Table 4). Formulation B was the only one showing lower average adsorption values than observed previously. These findings established the stability of the present composition to a drying process involving heat. Additionally, the use of a living bacteria of the same genus as the proteinaceous bacterial extract demonstrated comparable or lower detoxification efficacy compared to proteinaceous extract from bacterial fermentation from amino acid production alone (2). A biological detoxification activity is thus unlikely. Formulations C and D demonstrated again an advantage of the present composition toward deoxynivalenol sequestration.

TABLE 4
In vitro multi-mycotoxins adsorption characteristics of heat deactivated combinations of 2 sources
of yeast cell wall extract and clay material ([A, B] and [D, E]) admixed to 2 different
inclusion rates of proteinaceous extract from bacterial fermentation from amino acid production
([A, D] and [B, E]); composition (2) was a proteinaceous extract from bacterial fermentation
from amino acid production alone compared to the present composition of the original formulation
((1) aka., C)); and composition (5) a non-deactivated and propagated bacteria from Corynebacterium
genus, at pH 3.0 for two mixtures of mycotoxins commonly found in feedstuffs.

| | Percent Adsorption/Sequestration/Binding (%) | | | | | | | |
|---|---|---|---|---|---|---|---|---|
| Mycotoxin | Initial | | | Comp C | | | | |
| Mixtures | (ng/ml) | Comp A | Comp B | (aka. (1)) | Comp D | Comp E | Comp (2) | Comp (5) |
| Mycotoxin | 500 | 46.8 | 3.1 | 11.1 | 38.4 | 13.8 | 20.3 | 13.1 |
| Mixture A | | | | | | | | |
| Fumonisin B1 | | | | | | | | |
| Roquefortine C | 10 | 53.7 | 36.3 | 48.8 | 56.2 | 49.4 | 47.5 | 54.0 |
| Deoxynivalenol | 200 | 62.1 | 60.4 | 75.7 | 67.5 | 64.1 | 72.0 | 59.4 |
| T2-toxin | 20 | 0.0 | 0.0 | 2.6 | 3.3 | 9.1 | 16.6 | 13.5 |
| Aflatoxin B1 | 50 | 96.6 | 92.5 | 86.6 | 93.6 | 90.2 | 44.8 | 37.9 |
| Zearalenone | 20 | 49.7 | 29.0 | 45.8 | 59.4 | 47.8 | 60.9 | 43.5 |
| Ochratoxin A | 10 | 66.0 | 54.9 | 63.4 | 67.5 | 63.9 | 69.9 | 62.6 |
| Fusaric acid | 60 | 38.0 | 43.9 | 58.9 | 51.5 | 50.5 | 64.7 | 5.8 |
| Average Adsorption (%) | | 51.6 | 40.0 | 49.1 | 54.7 | 48.6 | 49.6 | 36.2 |
| Mycotoxin | 1500 | 55.8 | 5.4 | 18.2 | 41.6 | 17.0 | 25.2 | 15.4 |
| Mixture B | | | | | | | | |
| Fumonisin B1 | | | | | | | | |
| Roquefortine C | 10 | 60.7 | 31.6 | 51.7 | 59.3 | 48.3 | 49.8 | 50.9 |
| Deoxynivalenol | 500 | 61.8 | 58.9 | 76.1 | 65.7 | 64.1 | 72.1 | 54.7 |
| T2-toxin | 30 | 16.9 | 8.6 | 24.4 | 22.1 | 22.3 | 35.9 | 26.2 |
| Aflatoxin B1 | 50 | 98.1 | 90.5 | 90.1 | 95.0 | 96.4 | 60.3 | 50.1 |
| Zearalenone | 40 | 66.2 | 36.6 | 57.8 | 65.9 | 54.8 | 67.2 | 61.3 |
| Ochratoxin A | 50 | 94.6 | 72.7 | 89.5 | 95.3 | 88.9 | 95.3 | 86.0 |
| Fusaric acid | 120 | 73.1 | 68.9 | 86.6 | 74.6 | 76.7 | 83.0 | 44.0 |

| | | | | | | | |
|---|---|---|---|---|---|---|---|
| Average Adsorption (%) | 65.9 | 46.6 | 61.8 | 64.9 | 58.6 | 61.1 | 48.6 |
| Total Average Adsorption | 58.8 | 43.3 | 55.5 | 59.8 | 53.6 | 55.3 | 42.4 |
| (%) | | | | | | | |

### Example 4. Testing the Adsorption Capacity of the Composition for a Higher Titer of Mycotoxins in a Mixture

The present example is directed to the testing of the performance of the present compositions comprising a yeast cell wall extract and clay material admixed to a proteinaceous extract from bacterial fermentation from amino acid production mixture toward its capability at interacting with an iso-mixture of mycotoxins (e.g., Mycotoxin Mixtures C and D) each present at a concentration of 1,000 ng/ml in a citrate buffer environment of pH 3.0.

The same procedures as in Examples 1 to 3 were followed for the preparation and analysis of the different tested samples. In some embodiments, the inclusion levels of proteinaceous extract from bacterial fermentation from amino acid production was between 10 and 30%. The compositions used were heat inactivated prior to testing. The mycotoxins were used in the assay at 1,000 ng/ml each in a final reaction volume (e.g., Mycotoxin Mixtures C and D). The mycotoxins included in the assays and/or mixtures were selected from the common/regular and/or regulated mycotoxins category including aflatoxin B1, fumonisins B1, deoxynivalenol, T2-toxin, zearalenone, and emerging Fusarium mycotoxins category, such as fusaric acid, enniatins, beauvericin; Penicillium toxins such as roquefortine C, mycophenolic acid and citreoviridin; Aspergillus mycotoxins such as citrinin; and other mycotoxins of interest such as phomopsin A.

Using a higher concentration level of each mycotoxin (1000 ng/ml each) compared to Examples 1 to 3, in an iso-mixture condition (e.g., Mycotoxin Mixtures C and D), demonstrated that at higher concentration titers, the efficacy of adsorption of mycotoxins previously recorded was maintained or even improved (Table 5). An optimum efficacy was observed when using low inclusion rates of the proteinaceous bacterial extract in combination with yeast cell wall extract and clay material, further accentuated what seems to be a coalistic or synergic effect of the mixed composition toward deoxynivalenol, with adsorption percentage reaching about 62% and a dramatic increase of the fusaric acid sequestration at about 98%. Notable unexpected differences compared to previous results were observed, with an increase of T2-toxin sequestration between from about 51% to about 57% compared to about 20% previously recorded at lower mycotoxin concentrations. Fumonisin B1 displayed an improved adsorption rate at about 45%.

At the concentration used herein of 1,000 ng/ml of each mycotoxins, aflatoxin B1, roquefortine C and zearalenone exhibited lower percent adsorption than previously recorded, with percent values at about 66%, about 20 to about 28%, and about 19% to about 29%, respectively. These concentrations represent a large excess compared to normal mycotoxin contamination levels encountered in feedstuffs for those three mycotoxins that may invoke a saturation effect and hence the lower adsorption measured.

The results of the tested compositions for emerging mycotoxins adsorption showed between about 50% to about 60% sequestration activity toward beauvericin and enniatins emerging toxins but none for citreoviridin. These results diverged from previously observed interactions of traditional composition (4), where enniatin and beauvericin were sequestered from about 60% to about 100%, respectively, while citreoviridin was about 20%. Conversely, unexpected higher adsorption rates were observed with present compositions (1) and J (Example 1) for citrinin and phomopsin A with adsorption rates of about 57% to about 100% compared to historical composition (4) with less than about 36%.

TABLE 5
In vitro multi-mycotoxins adsorption characteristics of two heat
deactivated combinations of yeast cell wall extract and clay
material admixed to a proteinaceous extract from bacterial fermentation
from amino acid production with a high and low inclusion level
of the proteinaceous bacterial extract, at pH 3.0 for an iso-
mixture of regular and emerging mycotoxins.

| | | Percent Adsorption/Sequestration/ | |
|---|---|---|---|
| Mycotoxin | | Binding (%) | |
| Mixtures | Initial | Comp C (aka. | |
| C & D | (ng/ml) | (1)) | Comp J |
| Mycotoxin Mix C | 1000 | 45.3 ± 2.4 | 44.0 ± 2.1 |
| Fumonisin B1 | | | |
| Roquefortine C | 1000 | 19.9 ± 0.3 | 27.5 ± 2.4 |
| Deoxynivalenol | 1000 | 31.2 ± 1.0 | 61.7 ± 2.7 |
| T2-toxin | 1000 | 50.7 ± 1.8 | 56.6 ± 2.2 |
| Aflatoxin B1 | 1000 | 67.5 ± 2.0 | 65.7 ± 5.3 |
| Zearalenone | 1000 | 28.7 ± 2.2 | 18.7 ± 0.5 |
| Ochratoxin A | 1000 | 61.2 ± 0.9 | 59.3 ± 6.4 |
| Fusaric acid | 1000 | 98.4 ± 0.1 | 97.7 ± 0.4 |
| Average Adsorption (%) | | 50.4 ± 1.3 | 53.9 ± 2.8 |
| Mycotoxin Mix D | 1000 | 40.9 ± 4.6 | 50.6 ± 1.1 |
| Beauvericin | | | |
| Citreoviridin | 1000 | 1.9 ± 1.6 | 15.1 ± 4.3 |
| Citrinin | 1000 | 100.0 ± 9.6 | 78.0 ± 9.8 |
| Enniatin B | 1000 | 70.4 ± 12.3 | 60.7 ± 3.4 |
| Enniatin A | 1000 | 46.0 ± 10.6 | 56.5 ± 3.7 |
| Mycophenolate | 1000 | 33.6 ± 11.7 | 19.1 ± 4.9 |
| Phomopsin A | 1000 | 57.0 ± 13.7 | 73.5 ± 0.8 |

| | | |
|---|---|---|
| Average Adsorption (%) | 50.0 ± 7.8 | 50.5 ± 4.0 |

### Example 5. Establishing the Kinetic of Interaction of the Invention Compositions Toward Increasing Titers of Mycotoxins

This example is directed to testing of the performance of the inventive compositions comprising a yeast cell wall extract and clay material admixed to a proteinaceous extract from bacterial fermentation from amino acid production mixture toward its capability at interacting with an iso-mixture of mycotoxins each over a kinetic ranging from 500 to 5000 ng/ml in a citrate buffer environment at either pH 3.0 or pH 6.0, as respective conditions of the proximal and distal a digestive tract in monogastric and a rumen of ruminant animals.

The same procedures as in Examples 1˜4 were followed for the preparation and analysis of the different tested samples. Inactivation of the composition was performed using an autoclave set up at a temperature of 121° C. for 40 minutes. Six concentrations of the iso-mixtures were tested, at 500, 1000, 2000, 3000, 4000 and 5000 ng/ml or 0.5, 1.0, 2.0, 3.0, 4.0, 5.0 parts-per-millions (ppm).

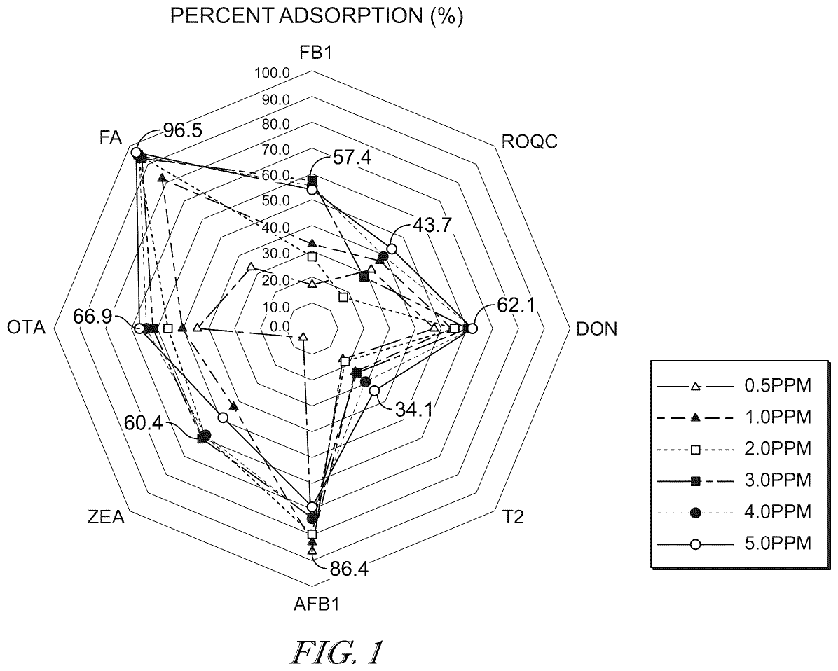

FIG. 1 demonstrates the present compositions over mycotoxins concentrations kinetic, high adsorption affinity for deoxynivalenol (DON, about 30% to about 62%) and fusaric acid (FA, about 34% to 95%), along with zearalenone (ZEA, from about 43% to about 60%) and ochratoxin A (OTA, about 45% to about 64%) when adsorption was performed at pH 3.0, the affinity increasing dose-dependently with higher titers of mycotoxins. Percent adsorption for aflatoxin B1 (AFB1) ranked from about 69% to about 98%, with decreasing concentrations of the mycotoxin, showing as saturation effect at higher concentrations. Percent adsorption for T2-toxin was at about 34% % at the higher concentrations of mycotoxins (e.g., above 5000 ng/ml). The invention composition also showed high affinity for fumonisin B1 (FB1) and roquefortine C (ROQC), but only for higher concentrations of mycotoxins above 3000 ng/ml, reaching about 50%.

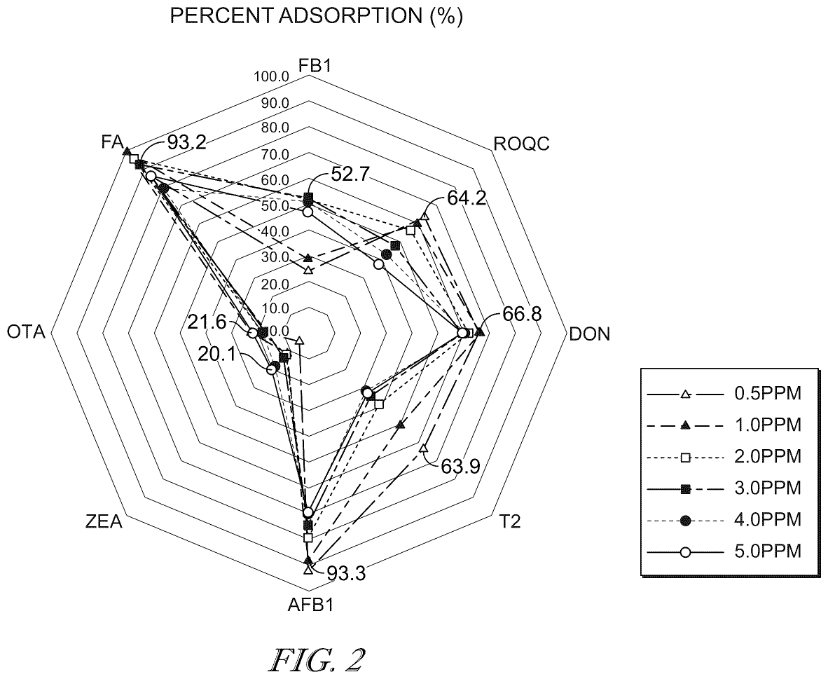

At pH 6.0 (see FIG. 2), the present compositions performed better than at pH 3.0 for all mycotoxins with the exception of zearalenone and ochratoxin. For those specific mycotoxins, an important drop in affinity was observed. This decrease was also observed for historical composition embodiment (4).

### Example 6. Viability Measurement of Intestinal Cells after Exposure to Deoxynivalenol and/or Fusaric Acid in the Presence or Absence of the Present Inventive Composition

The in vitro evaluation of the incidence of deoxynivalenol and/or fusaric acid mycotoxins present, either individually or in mixture, on the viability of animal cells, such as intestinal porcine epithelial-jejunal cells (IPEC-J2), was measured in the presence or absence of the present compositions. The goal was to demonstrate an increase of the viability of the IPEC-J2 cell system in the presence of the present compositions.

IPEC-J2 cells were seeded in 96-well plates (10,000 cells/well) and grown to 80% confluence overnight. Cells received fresh media with diluted sample and incubated over a 24-hour exposure period.

Individual mycotoxins, deoxynivalenol, fusaric acid, or a combination of deoxynivalenol and fusaric acid (1:1, volume-by-volume) were reacted together with the present compositions using the same adsorption reactions described in Examples 1 to 5. The mycotoxins were reacted with the present composition in a 10 mM ammonium citrate buffer creating a 0.2% weight-by-volume inclusion of the compositions as a slurry in buffer. Under continuous mixing, the slurry was transferred to pre-siliconized Pierce™ Spin columns with end caps on for each mycotoxin type and concentration to be tested. Reactions were performed under orbital shaking at 37° C. for 1 hour.

Columns were uncapped, placed into pre-siliconized 2 mL collection tubes, and centrifuged at 2,000 RPM for 2 minutes to collect filtrate. Filtrates were diluted with complete media to desired DON concentrations (0-10 ppm for the control without the invention compositions) and applied directly to the 96 well plated seeded with IPEC-J2 cells over a 24-hour incubation. Viability was measured by MTT assay: MTT solution (5 mg/mL in PBS) was diluted in serum free media (DMEM 1×) to a final concentration of 0.5 mg/mL. Cells were rinsed once with PBS and incubated with reagent mixture for 4 hours. A separate 96-well plate containing no cells was prepared and treated simultaneously for background measurement. Formazan formations were extracted and solubilized by 30-minute incubation/agitation with pre-warmed DMSO. Optical density was measured by means of a plate reader (Biotek Synergy H1 microplate reader) at 590 nm wavelength and used to calculate percent viabilities (relative to untreated control).

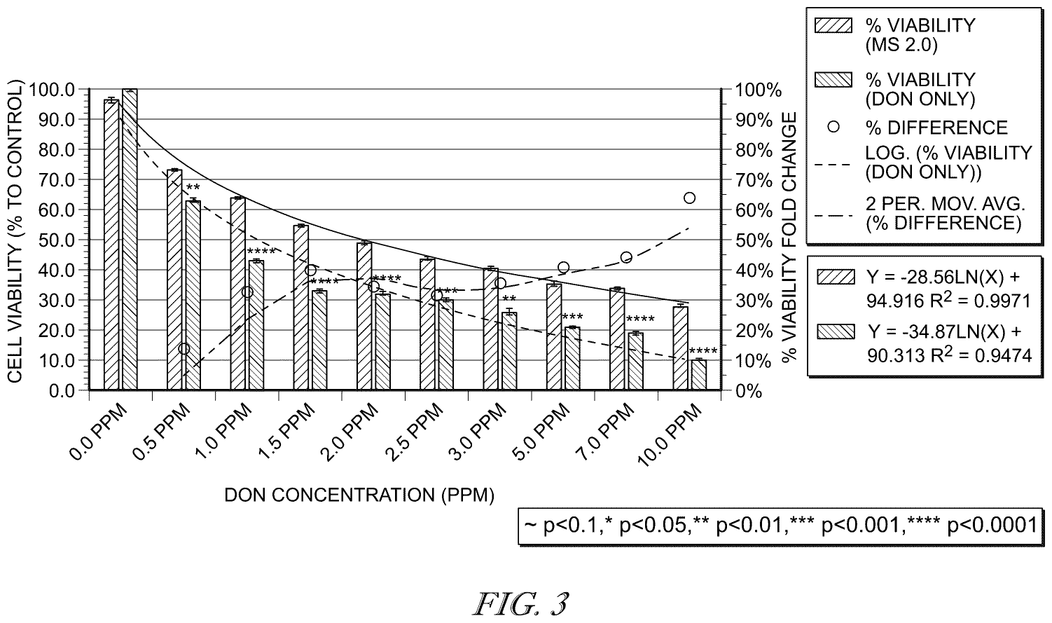

The viability measurement of IPEC-J2 cells after 24-hour exposure to deoxynivalenol showed a decrease in cell viability with increased titers of the toxin (from 0 ppm to 10 ppm or μg/mL). The addition of the present inventive compositions significantly (p<0.01) mitigated those effects and improved in a dose-dependant fashion the relative cell viability from about 20% for lower mycotoxin titer to 65% for the highest mycotoxin concentration (see FIG. 3). Fusaric acid displayed only a limited non-significant effect on cell viability (about 52% to 100%) at any concentrations up to 5.0 mg/kg.

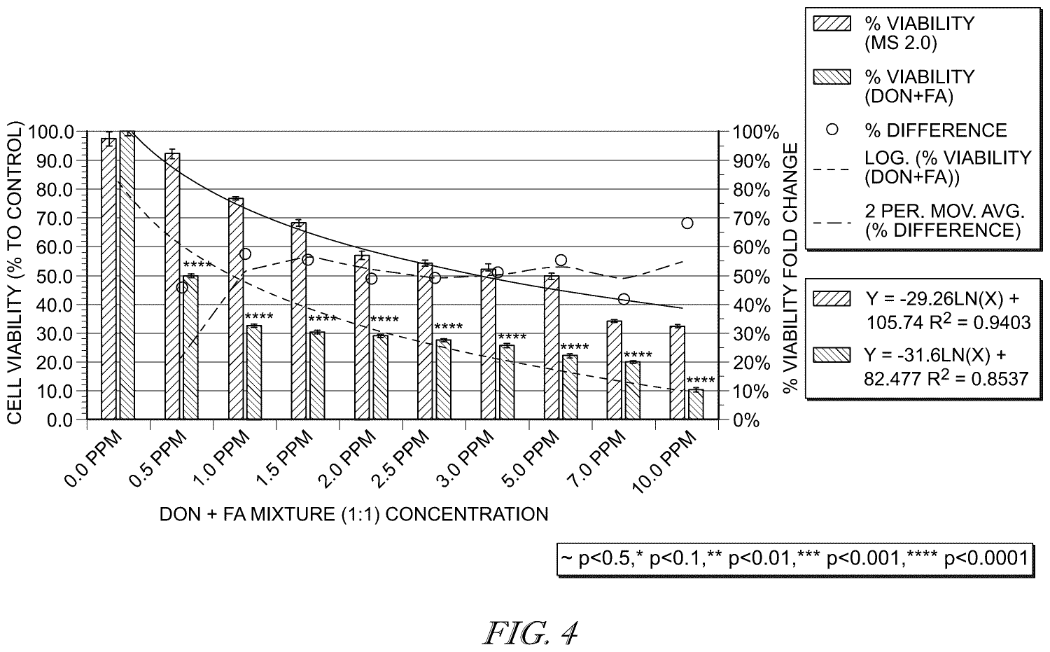

No differences were seen when the present composition was used. The combination of deoxynivalenol and fusaric acid (1:1, v/v) showed a more pronounced decrease in relative viability with increased titers of the mycotoxin iso-mixture (from 0 ppm to 10 ppm or μg/mL). The application of the inventive compositions was able to significantly (p<0.0001) mitigate this effect and improved cell viability between from about 46% to about 68% (see FIG. 4). This denoted a synergistic activity at lower mycotoxin titers of the effect of the present invention compositions on the combination of deoxynivalenol and fusaric acid on cell relative viability.

### Example 7. In Vitro Efficacy of Sequestration of a Yeast Cell Wall Extract and Clay Material when Admixed to a Proteinaceous and/or Cell Wall Extract(s) from Bacterial Fermentation from Amino Acid Production, with or without Incorporation of Algal Material

Four compositions, (1) a yeast cell wall extract and clay material admixed to bacterial biomass rich in protein and (2) a yeast cell wall extract, a clay material and an algal material admixed to bacterial biomass rich in protein were tested and compared to historical compositions such as (3) yeast cell wall extract and clay material mixture and (4) a yeast cell wall extract and clay material admixed to algal material, were tested each at the same weight to volume ratio for their efficiency at interacting with two different mixtures of mycotoxins (Mycotoxin Mixtures A and B) in a citrate buffer environment of pH 3.0, as condition of the proximal digestive tract in monogastric animals.

A 0.1 g sample from compositions (1), (2), (3), or (4) was weighed and transferred into microcentrifuge tubes. To each tube, 1 ml of ammonium citrate buffer at pH 3.0 was added to create a suspension. A 24 μL portion of the slurry sample was then applied to a 576 μL mixture of mycotoxins. One embodiment of an isomixture of mycotoxins comprised 37 mycotoxins from Aspergillus, Fusarium, Penicillium and other fungal species that were each tested at a concentration of 1 μg/mL in the reaction media. This mixture was placed into a Pierce Spin Column cassette, which contained a frit and filter disc, to achieve a final adsorbent concentration of 0.4% weight by volume. This procedure was repeated for each replicate and product.

The samples were incubated for 60 minutes on a rocker platform set to a low speed, approximately 150 rpm, at 37° C. Following incubation, the cassettes were centrifuged at 10,000 rpm for 2 minutes. A 200 μL aliquot of the sample was transferred to a 1 mL silanized glass UPLC vial with low-volume recovery, prior to being injected into the UPLC-MS sampler for quantification. To account for potential signal suppression or enhancement during the mass spectrometry detection process, 13C-labeled internal standards for mycotoxins were added to the samples.

Mycotoxins were separated using a CORTECS reverse-phase C18 column (2.7×100 mm, 1.6 μm, Waters Corp.) set to 40° C. A gradient elution was performed with a dual mobile phase, comprising OptimaGrade water with 0.1% formic acid and methanol MS grade solvent with 0.1% formic acid, at a flow rate of 0.3 mL/min, utilizing an Acquity UPLC system (Waters Corp.). A volume of 10 μL of sample was injected in the system. The UPLC system was coupled with the IMS-QTOF-MSE mass spectrometer (Vion™, Waters Corp.) equipped with an electro-spray ionization (ESI) source, operating in positive and negative ion modes. The mass spectrometer parameters were optimized using standard procedures used by experienced operators.

The results representing the percent of adsorption of 33 exploitable mycotoxins investigated against a 1 μg/mL isomixture of mycotoxins are presented in Table 6. The adsorption percentage was determined by comparing control samples, which contained only the mycotoxins in buffer, with the free mycotoxins present in the supernatant of samples treated with the described compositions. Mycotoxin concentrations were derived from the integration of the area under the curve of chromatograms generated after UPLC separation and elution of the target analytes, as well as from the total ion chromatograms produced by mass spectrometry. Each sample's concentration was calculated by comparing the signal intensity detected by the mass spectrometer to the control analytical standard curves, which included a minimum of five concentration points, and were normalized using 13C-labeled mycotoxin internal standards.

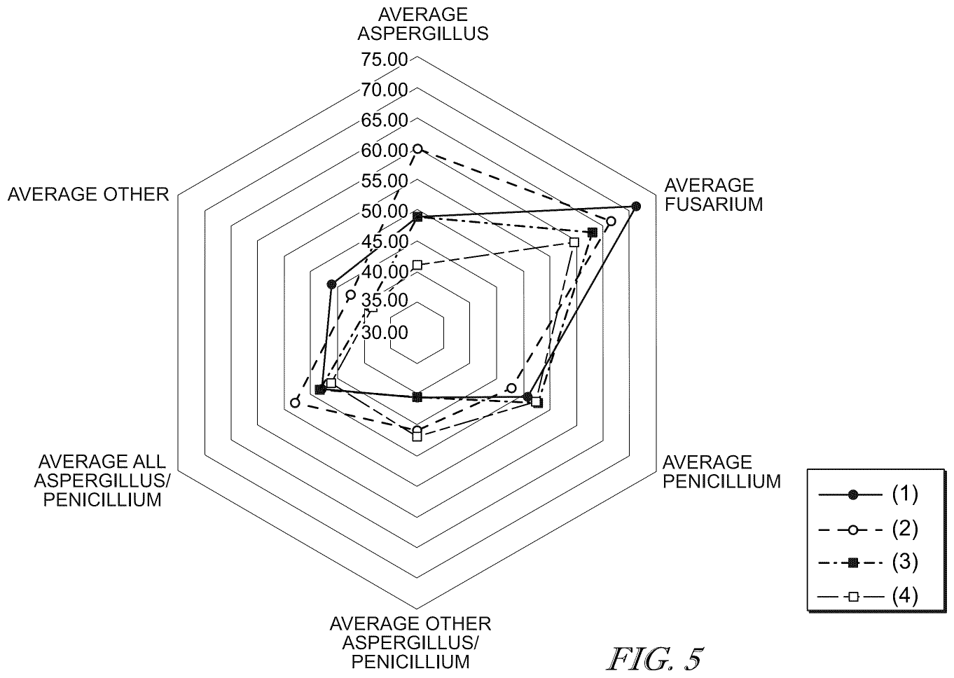

The data presented in Table 6 shows an increase in the binding efficacy of the inventive compositions when (1) a yeast cell wall extract and clay material was admixed to bacterial biomass rich in protein and when (2) an algal material was added to a yeast cell wall extract, a clay material further admixed to bacterial biomass rich in protein as compared to the historical formulations (3) and (4) consisting only of a yeast cell wall extract and clay material, respectively, with or without the addition of algal material. The composition samples were tested across 33 different mycotoxins for their adsorptive properties when they are present in an isomixture. The average total sequestration for the 33 mycotoxins tested was of about 54% and 55% for compositions (1) and (2) compared to 51% and 48% for historical compositions (3) and (4), respectively. The data of the categorical average adsorption rate per fungal mycotoxin producing species (e.g., Aspergillus, Fusarium, Penicillium and other fungal species) is presented in FIG. 5 and Table 7. Data showed that, as demonstrated in Examples 1-5, the admixing of a proteinaceous extract(s) from bacterial or fungal fermentation from amino acid production contributed to the improvement of the adsorption capabilities recorded for certain mycotoxin of interest.

Specifically, significant positive differences over the other formulations were seen for the sequestration of aflatoxin with an average adsorption of 98%; 70% for ochratoxin A; 30% for patulin and satratoxin, by present composition (1) where yeast cell wall extract and clay material was admixed to bacterial biomass rich in protein. However, composition (2) with the addition of algal material provided a significant increase of the adsorption for citrinin, with 88% of the toxin bound compared with less than 44% with other formulations; 74% of penitrem A; and 46% of alteroxin-I. Both compositions (1) and (2) showed an improvement in adsorption capabilities for deoxynivalenol reaching respectively about 47% and 53% compared to about 29% and 20% for (3) and (4), which represented respectively a 161% and 259% increase in sorption ability. Similarly, compositions (1) and (2) significantly improved the sequestration capabilities toward fusaric acid with about 72% and 79% toxin bound compared to 58% and 47%, respectively for historical compositions (3) and (4), accounting for an increase of 124% and 168% respectively. Both compositions (1) and (2) also had beneficial features over the sequestration of ochratoxin A, T-2 toxin, aurofusarin, mycophenolic acid, alternariol, tentoxin, while preserving high adsorptive efficacy features toward aflatoxins, beauvericin, enniatins, ochratoxin A, penicillic acid, paxillin, alternariol, cyclopiazonic acid and t a lesser extent, chaetocin, fumagillin, sporidesmin, and fumitremorgin C.

Overall, these results confirmed the important breakthrough and synergetic potential of the combination of a bacterial biomass rich in protein to a yeast cell wall and clay composition, as provided in the present inventive composition. The additional presence of algal material in the present composition, for some toxins, further increased the functionality of the present composition. This also demonstrated coalistic and/or synergic effects of the combination of ingredients of the present composition to further improve the detoxification activity toward toxins that have been, chemically and stereochemically, difficult to interact.

TABLE 6
In vitro multi-mycotoxins adsorption characteristics of compositions: (1) a yeast cell wall extract
and clay material admixed to bacterial biomass rich in protein, (2) a yeast cell wall extract,
a clay material and an algal material admixed to bacterial biomass rich in protein, (3) a yeast
cell wall extract and clay material mixture and (4) a yeast cell wall extract and clay material
admixed to algal material evaluated at pH 3.0 toward a mycotoxin isomixture at a concentration
of 1.0 μg/mL. Results were expressed as average binding percentage (%). Student t test, Means
within a row with no common letter indicate significant differences, p ≤ 0.05.

| | | Percent Adsorption/Sequestration/Binding (%) | | | |
|---|---|---|---|---|---|
| Fungal species | Mycotoxins | Comp (1) | Comp (2) | Comp (3) | Comp (4) |
| Aspergillus spp. | Aflatoxin B1 | 97.9 ± 0.2^{a} | 93.4 ± 4.3^{ab} | 96.2 ± 1.0^{b} | 95.5 ± 1.0^{b} |
| | Citrinin | 43.0 ± 6.3^{a} | 87.8 ± 2.9^{b} | 43.5 ± 11.6^{a} | 36.1 ± 9.8^{a} |
| | Cytochalasin A | 27.4 ± 2.3^{a} | 84.9 ± 7.9^{b} | 24.9 ± 3.8^{a} | 24.9 ± 3.1^{a} |
| | Fumigalin | 50.1 ± 3.6^{a} | 52.5 ± 3.9^{a} | 54.2 ± 3.6^{a} | 41.4 ± 3.5^{b} |
| | Myriocin | 37.5 ± 1.0^{a} | 33.3 ± 5.5^{a} | 32.3 ± 4.4^{a} | 11.1 ± 2.6^{b} |
| | Ochratoxin A | 70.1 ± 0.3^{a} | 65.8 ± 3.6^{ab} | 64.0 ± 3.5^{b} | 52.4 ± 1.6^{c} |
| | Verruculogen A | 34.1 ± 4.8^{a} | 33.7 ± 6.2^{a} | 51.8 ± 2.8^{b} | 50.9 ± 7.1^{b} |
| | Verruculogen J | 30.9 ± 2.5^{a} | 29.0 ± 2.4^{ab} | 24.4 ± 3.0^{b} | 17.0 ± 3.1^{c} |
| Fusarium spp. | Deoxynivalenol | 46.8 ± 2.9^{a} | 53.3 ± 3.9^{a} | 29.1 ± 5.0^{b} | 20.6 ± 4.2^{b} |
| | T-2 toxin | 24.1 ± 0.2^{a} | 20.7 ± 6.2^{a} | 1.7 ± 3.2^{b} | 5.3 ± 2.3^{b} |
| | Beauvericin | 95.0 ± 5.7^{ab} | 95.1 ± 1.2^{a} | 98.2 ± 1.4^{b} | 98.2 ± 0.6^{b} |
| | Enniatin A | 94.2 ± 1.1^{a} | 71.0 ± 7.9^{b} | 95.3 ± 0.9^{a} | 94.2 ± 1.8^{a} |
| | Enniatin B | 95.0 ± 2.0^{a} | 64.7 ± 2.3^{b} | 97.2 ± 0.2^{a} | 96.2 ± 0.4^{a} |
| | Fusaric acid | 72.3 ± 3.3^{a} | 78.9 ± 3.4^{a} | 58.2 ± 2.5^{b} | 47.0 ± 2.0^{c} |
| | Aurofusarin | 93.4 ± 0.9^{a} | 92.8 ± 1.7^{a} | 67.1 ± 8.4^{b} | 65.9 ± 4.8^{b} |
| | Zearalenone | 47.5 ± 5.9 | 54.4 ± 2.3 | 55.6 ± 4.5 | 48.4 ± 4.4 |
| Penicillium spp. | Griseofulvin | 35.6 ± 2.2^{a} | 32.8 ± 6.7^{ab} | 31.5 ± 3.4^{ab} | 29.1 ± 1.9^{b} |
| | Mycophenolic | 33.6 ± 0.9^{a} | 27.6 ± 5.3^{a} | 6.4 ± 3.9^{b} | 6.2 ± 2.0^{b} |
| | acid | | | | |
| | Paxilline | 68.5 ± 2.6^{a} | 74.9 ± 1.1^{b} | 84.7 ± 1.3^{c} | 70.9 ± 1.9^{a} |
| | Penicillic acid | 80.5 ± 3.2^{a} | 74.8 ± 4.3^{a} | 78.6 ± 0.6^{b} | 100.0 ± 1.8^{c} |
| | Roquefortin C | 38.8 ± 0.4^{a} | 33.3 ± 5.5^{a} | 60.0 ± 1.3^{b} | 55.9 ± 0.5^{c} |
| | Wortmannin | 47.5 ± 4.3^{a} | 43.3 ± 7.3^{a} | 55.4 ± 2.1^{b} | 51.3 ± 2.9^{ab} |
| Aspergillus/ | Cyclopiazonic acid | 61.6 ± 6.8 | 63.0 ± 7.8 | 68.4 ± 12.0 | 75.8 ± 1.9 |
| Penicillium | Fumitremorgin C | 50.1 ± 3.6^{ab} | 52.5 ± 3.9^{a} | 54.2 ± 3.6^{a} | 41.4 ± 3.5^{b} |
| | Patulin | 30.4 ± 3.4^{a} | 18.7 ± 3.9^{b} | 16.2 ± 5.1^{b} | 0.0 ± 20.6^{bc} |
| | Penitrem A | 58.0 ± 5.1^{a} | 73.6 ± 2.5^{b} | 56.3 ± 3.7^{a} | 69.6 ± 1.7^{b} |
| Alternaria spp. | Alternariol | 88.1 ± 2.9^{a} | 88.7 ± 3.2^{a} | 87.5 ± 3.9^{b} | 72.0 ± 0.8^{b} |
| | Altertoxin-I | 30.7 ± 10.0^{ab} | 46.0 ± 4.7^{a} | 19.3 ± 29.4^{ab} | 34.6 ± 3.0^{b} |
| | Tentoxin | 11.4 ± 1.5^{a} | 10.4 ± 3.5^{a} | 0.0 ± 0.0^{b} | 17.0 ± 3.8^{a} |
| Chaetomium spp. | Chaetocin | 63.2 ± 10.5^{abc} | 65.6 ± 2.8^{a} | 75.4 ± 2.8^{b} | 51.0 ± 2.4^{c} |
| Stachybotrys spp. | Satratoxin | 30.5 ± 0.9^{a} | 23.0 ± 4.4^{b} | 0.0 ± 0.0^{c} | 7.8 ± 2.3^{d} |
| Pythomyces spp. | Sporidesmin | 52.6 ± 0.7^{a} | 21.3 ± 4.4^{b} | 51.9 ± 1.5^{a} | 48.1 ± 1.5^{c} |

| | | | | |
|---|---|---|---|---|
| Total Average Adsorption (%) | 54.4 ± 3.2^{a} | 55.0 ± 4.3^{a} | 51.2 ± 4.2^{ab} | 48.0 ± 4.2^{b} |

TABLE 7
In vitro categorical adsorption characteristics according to fungal mycotoxin-
producing species for compositions: (1) a yeast cell wall extract and clay material
admixed to bacterial biomass rich in protein, (2) a yeast cell wall extract,
a clay material and an algal material admixed to bacterial biomass rich in protein,
(3) a yeast cell wall extract and clay material mixture and (4) a yeast cell
wall extract and clay material admixed to algal material evaluated at pH 3.0
toward a mycotoxin isomixture at a concentration of 1.0 μg/mL. Results were
expressed as average binding percentage (%). Student t test, Means within a row
with no common letter indicate significant differences, p ≤ 0.05.

| | Average Percent Adsorption/Sequestration/Binding (%) | | | |
|---|---|---|---|---|
| Mycotoxin fungal categories | Comp (1) | Comp (2) | Comp (3) | Comp (4) |
| Aspergillus toxins | 48.9 ± 2.6^{a} | 60.1 ± 4.6^{b} | 48.9 ± 4.2^{ac} | 41.2 ± 4.0^{c} |
| Fusarium toxins | 71.0 ± 2.7^{a} | 66.4 ± 3.6^{bc} | 62.8 ± 3.3^{c} | 59.5 ± 2.6^{c} |
| Penicillium toxins | 50.7 ± 2.3^{a} | 47.8 ± 5.0^{a} | 52.8 ± 2.1^{a} | 52.2 ± 1.8^{b} |
| Other Aspergillus/ | 40.6 ± 11.0 | 45.9 ± 4.8 | 40.4 ± 6.7 | 46.9 ± 6.7 |
| Penicillium toxins | | | | |
| All Aspergillus/ | 47.7 ± 4.4^{a} | 52.8 ± 4.8^{b} | 48.3 ± 4.1^{a} | 46.1 ± 3.9^{a} |
| Penicillium toxins | | | | |

### Example 8. In Vitro Effect of the Digestive Extract of Yeast Cell Wall Extract and Clay Material when Admixed to a Proteinaceous and/or Cell Wall Extract(s) from Bacterial Fermentation from Amino Acid Production on the IPEC-J2 Cell Viability

The inventive composition (1) a yeast cell wall extract and clay material admixed to bacterial biomass rich in protein was tested in a preliminary experiment for its capability at maintaining or improving cell viability of intestinal porcine epithelial intestinal cells of the jejunum (IPEC-J2), after undergoing digestive conditions of the digestive intestinal tract. The IPEC-J2 cells were also cultivated in the presence or absence of DON as a differential evaluation to the application of the inventive composition.

A sequential digestion protocol was applied to the composition invention encompassing: (i) oral digestion model: 1 g of sample was added to a 1 mL solution of to a simulated salivary fluid (SSF, 12 mM KH₂PO₄, 40 mM NaCl, added with 1.5 mM CaCl₂), adjust to pH 7.0 with NaOH) containing 150 U/ml of porcine alpha amylase; (ii) gastric digestion model: the oral digesta was further diluted to 2 mL of a gastric solution made of 2,000 U/ml porcine pepsin in simulated gastric fluid (SGF, 0.2% (w/v) NaCl, 0.7% (v/v) HCl, added with 1.5 mM CaCl₂), pH 2.0) incubated for 2 h at 37° C. under agitation; (iii) intestinal digestion model: the gastric digesta was diluted to 4 mL of a digestion solution made of a 100 U/ml of pancreatin into a simulated intestinal fluid (SIF, 12 mM KH₂PO₄, pH to 8.0 with NaOH, added with 0.3 mM CaCl₂), pH 8.0) and incubated for 2 h at 37° C. under agitation. Bile salts can be optionally added at a 10 mM concentration.

The reaction was stopped by heating the solution at 90° C. for 10 minutes. The final digesta was then centrifuged and the digested inventive composition supernatant was incubated with the IPEC-J2 cells added to the cell culture medium at 2 mg/ml inclusion rate. The deoxynivalenol mycotoxin was added to a separate set of cells in the complete media to a final concentration of 5.0 μg/mL. The growth rate and proliferation were compared to the growth rate of a control set of cells receiving only the cell growth media without mycotoxin addition or invention composition digesta supplementation.

IPEC-J2 cells were seeded in 25 cm² flasks overnight and grown to approximately 80% confluency (estimate of 1.0-2.0×10⁻⁶ cells per flask). Cells received fresh media with diluted sample and incubated over a 24-hour exposure period. Trypan blue was used to directly visualize and quantify dead cells via staining of damaged membranes.

The visual viability measurement of IPEC-J2 cells after 24-hour exposure to either the inventive composition (1) digesta, DON compared to non-treated cells, showed an increase in total cell counts with the yeast cell wall extract and clay material admixed to bacterial biomass rich in protein (Table 8). The new composition increased the number of living cells compared to both control and DON treated cells by around +20%. The deoxynivalenol treatment decreased the number of living cells compared to the control, demonstrating the toxicity of the mycotoxins and reduced viability by around-18%.

The beneficial effects observed in this example from the inventive composition of the present disclosure were directly related to a direct effect of the digested material itself on cellular viability. This is different from the previously observed mitigation effects that were only linked to a decrease of the bioavailability of the deoxynivalenol and fusaric acid mycotoxins by means of adsorption/sequestration; where toxins were separately interacted with a non-digested product, and where the remaining free toxins present in the media post-reaction were collected and applied to the IPEC-J2 cells for viability testing. Thus, we can conclude that in reality, the present composition could have a combined effect on the direct interaction with mycotoxins that can limit their bioavailability and further absorption by the intestinal cells and a protective effect of the inventive composition itself on mycotoxin cell survival capacity.

TABLE 8
In vitro viability assay using IPEC-J2 cells cultured with a
composition (1) yeast cell wall extract and clay material
admixed to bacterial biomass rich in protein or DON at 5.0 μg/
mL compared to control treatment with cell culture growth media.
Viability (live/dead) IPEC-J2 Trypan Blue Counts

| Treatment | Live | Dead | Total | Live/Dead |
|---|---|---|---|---|
| (1), 2 mg/mL | 1.63 × 10\hat{ }6 | 4.19 × 10\hat{ }4 | 1.67 × 10\hat{ }6 | 97%/3% |
| DON, 5.0 μg/mL | 1.11 × 10\hat{ }6 | 4.71 × 10\hat{ }4 | 1.16 × 10\hat{ }6 | 96%/4% |
| Control (no | 1.36 × 10\hat{ }6 | 2.62 × 10\hat{ }4 | 1.38 × 10\hat{ }6 | 98%/2% |
| treatment) | | | | |

### Example 9. In Vivo Efficacy of Sequestration of a Yeast Cell Wall Extract and Clay Material when Admixed to a Proteinaceous and/or Cell Wall Extract(s) from Bacterial Fermentation from Amino Acid Production

To assess the efficacy of the inventive composition in living animals, an experimental trial was constructed to determine the beneficial effect of the present composition comprising a yeast cell wall extract and clay material admixed to bacterial biomass rich in protein supplemented to the diet of broiler chicken. The broiler chickens were subjected to a natural Fusarium mycotoxin challenge and compared to animals not supplemented with the inventive composition or subjected to a mycotoxin challenge. The objectives were to evaluate the impact on performance, immune status, and liver integrity in broiler birds subjected to the natural multi-mycotoxin challenge. The measure of the mycotoxins levels in blood would inform of the protective effect of the present composition using differential analysis to measure the effectiveness of the mitigation strategy. Mycotoxins were selected according to the specific augmented functionalities toward deoxynivalenol in particular, in combination with zearalenone. The examination of various inclusion levels of the composition supplemented to the diet could inform on the optimal dosage.

A pre-trial evaluation was performed to locate a naturally contaminated source of distiller dry grains (DDGS), which was sourced and evaluated after solvent extraction using liquid chromatography coupled to mass chromatography analysis, and determined to contain quantities of around 25 mg/kg of deoxynivalenol, 2.0 mg/kg of zearalenone, and 1.5 mg/kg of fusaric acid. A second DDGS sample was also located containing marginal levels of mycotoxins. Both DDGS materials were used at an inclusion rate of about 25% in feedstuffs to produce one non-contaminated and six contaminated complete diets, divided in two feeding phases, starter and grower feed material. Quantitative analysis of the feedstuffs (starter and grower diets) was performed and is summarized in Table 9. Final levels of deoxynivalenol averaged about 8.0 mk/kg of feed and zearalenone around about 0.6 and about 0.1 mg/kg of starter and grower feed, respectively. All other mycotoxins found in the diet were present at a marginal concentration.

TABLE 9
Mycotoxins contamination levels produced in feedstuffs using a
25% inclusion of DDGS intended for use as starter and growing
dietary phases in a challenge study performed in broiler chicken.

| | Average | Average | Average | Average | Average | Average |
|---|---|---|---|---|---|---|
| | Starter | Starter | Starter | Grower | Grower | Grower |
| Detected | (ug/kg) | (ug/kg) | (ug/kg) | (ug/kg) | (ug/kg) | (ug/kg) |
| Mycotoxins | T3 | T4 | T5 | T3 | T4 | T5 |
| Deoxynivalenol | 8086.70 | 7559.33 | 7906.70 | 6898.60 | 7239.20 | 7873.07 |
| 3-Acetyl-DON | 78.57 | 72.07 | 82.57 | 78.87 | 86.00 | 93.63 |
| 15-Acetyl- | 529.97 | 482.33 | 510.70 | 512.90 | 557.43 | 614.80 |
| DON | | | | | | |
| DON-3- | 724.33 | 725.53 | 848.37 | 278.90 | 311.60 | 331.57 |
| Glucoside | | | | | | |
| Beauvericin | 19.80 | 18.37 | 10.53 | 0.00 | 8.57 | 9.37 |
| Moniliformin | 58.70 | 37.70 | 0.00 | 0.00 | 104.47 | 113.37 |
| Enniatin A/A1 | 0.00 | 5.10 | 5.70 | 8.63 | 8.65 | 9.05 |
| Fusaric Acid | 148.50 | 159.80 | 149.10 | 154.60 | 188.43 | 206.93 |
| Fumonisin B1 | 234.23 | 190.23 | 250.20 | 725.60 | 260.13 | 238.40 |
| Fumonisin B2 | 50.47 | 0.00 | 40.53 | 85.67 | 0.00 | 0.00 |
| Fumonisin B3 | 14.13 | 15.37 | 14.03 | 57.37 | 17.43 | 20.90 |
| Zearalenone | 665.57 | 559.67 | 661.30 | 103.37 | 122.13 | 150.00 |

A pre-starter non-contaminated diet was used from Day 1 to 9, then the contaminated starter diets were used from Day 10 to 21 followed by the contaminated grower diet for the following 3 weeks of the trial. All birds were fed the same diet with good quality DDGs for pre-starter diet (1-9 days). The treatment diets were fed to chickens from Day 10 to 42. Typical broiler meal diets (particle size of 800-900 um) were provided on an ad libitum basis. The yeast cell wall extract and clay material admixed to bacterial biomass rich in protein was supplemented to three diets at 1.0, 2.0 and 4.0 kg/T of feed, while one treatment only used the contaminated diet without the inventive composition, and another did not contain the mycotoxin challenge nor the inventive composition at all (e.g., control).

Environmental factors, such as stocking density, temperature of the animal facility, lighting, humidity, light/dark cycle were recorded. The following performance parameters were measured in three phases (pre-starter phase (1-9 days), starter phase (10-21 days) and grower phase (22-42 days).

During the trial, samples of blood were collected in heparin tubes at Days 28 and 41. In addition caecal samples were collected from the digestive system of the animals into Biofreeze™ preservation media. In caecal samples, microbial DNA extraction was performed. Samples were washed to remove solid particles and complex polysaccharides that could interfere with DNA purification and qPCR.

The liquid phase underwent differential centrifugation to collect bacterial cells, which were then disrupted to extract and purify chromosomal DNA. The qPCR experiment was used to detect and quantifies fluorescent signals, which increase in proportion to the amount of PCR product in the reaction. By measuring fluorescence emission (using SYBR Green I) at each cycle, the reaction can be monitored during the exponential phase, where the initial increase in PCR product correlates with the starting amount of target template. Calprotectin levels were measured using a specific enzyme-linked immunosorbent assay (ELISA), and the results were normalized based on the weight of the original caecal sample. Short chain fatty acid profiles from samples were analyzed by gas chromatography using pivalic acid as an internal standard.

Chicken blood was collected on Day 28 and Day 41 with blood samples from each bird (pen) collected in duplicate on volumetrically accurate microsampling (VAMS) devices. The VAMS were dried and stored at 4° C. All VAMS tips were extracted with an organic solvent, water and acid mix spiked with 4 isotopically labelled mycotoxins used as internal standards for signal suppression or enhancement correction. Briefly, 300 μL of the extraction solution was added to each VAMS tip and were sonicated for 20 minutes, then placed on shaker for 30 minutes.

After conditioning approximately, 300 μL of blood extract was taken up in HybrindSPE DPx zirconium loaded pipet tips to remove interfering phospholipids from blood samples before being transferred into new Eppendorf tubes and dried. Samples were reconstituted using 60 uL of MS grade methanol/water/formic acid, 60/40/0.1% (v/v/v) solvent mixture. All samples were then centrifuged at 13,000 g for 5 minutes and supernatant was placed into total recovery UPLC vials and analyzed by UPLC-QTOF-MSE (Vion, Waters Corp). A 7-point mycotoxin calibration curve with spiked internal standard was used for quantification. Elution was performed in a gradient mode using water and methanol acidified with formic acid over a 15 min runtime. Samples were run in positive and negative electrospray ionization mode.

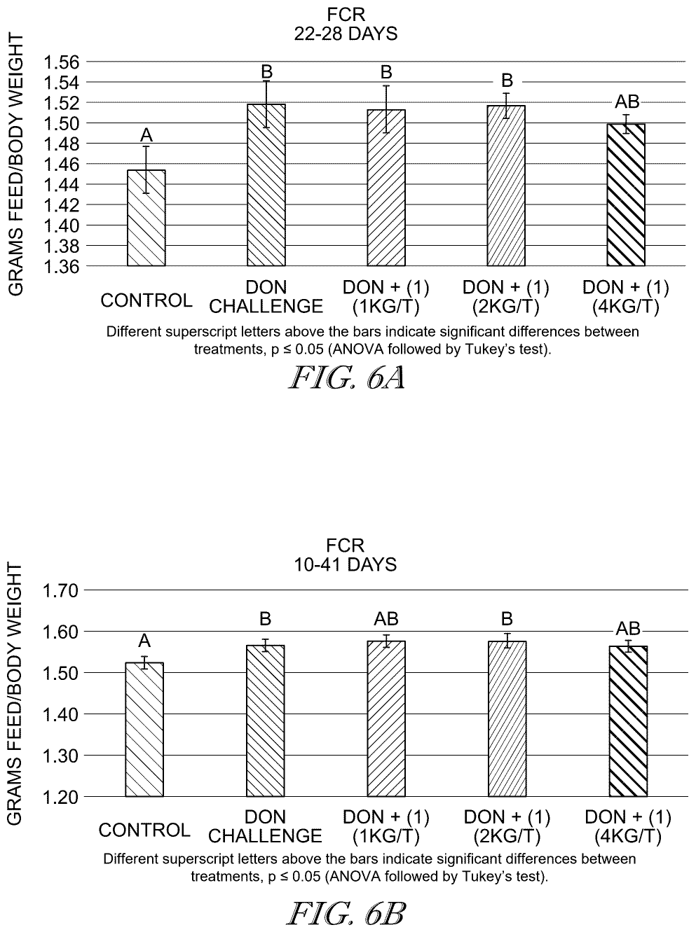

The zootechnical parameters, such as body weight gain, body weight gain and feed consumption did not provide significant difference according to the dietary treatments despite the observation of an effect from the mycotoxin challenge. As seen in FIG. 6A, between 22 and 28 days, a recovery of the feed conversion could be observed with the application of the inventive composition (e.g., (1)) at an inclusion rate of about 4 kg/T of feed. From 29 to 35 days, no feed conversion ratio (FCR) differences could be seen between treatments indicating a general recovery with maturity of the bird. Over the total experimental challenge period (10-41 days, FIG. 6B), a significant increase was observed in the FCR of the birds subjected to the challenge treatment only and a recovery with FCR levels was non-significant from the ones of the control for the 1.0 kg/T and 4.0 kg/T inclusion rate of the inventive composition, but not for the 2.0 kg/T inclusion rate.

Blood biomarker evaluations showed that calprotectin levels of the birds receiving the mycotoxin challenge only tended to decrease compared to control treatment receiving no mycotoxins. Trends toward a recovery was found when the (1) yeast cell wall extract and clay material admixed to bacterial biomass rich in protein was used at the original levels. As a biomarker of inflammation, this outcome could highlight the potential deregulation effect of the Fusarium mycotoxins present in the diet on the immune response, with a decrease of the response of white blood cells or a relocation of white blood cells to the intestinal tissues.

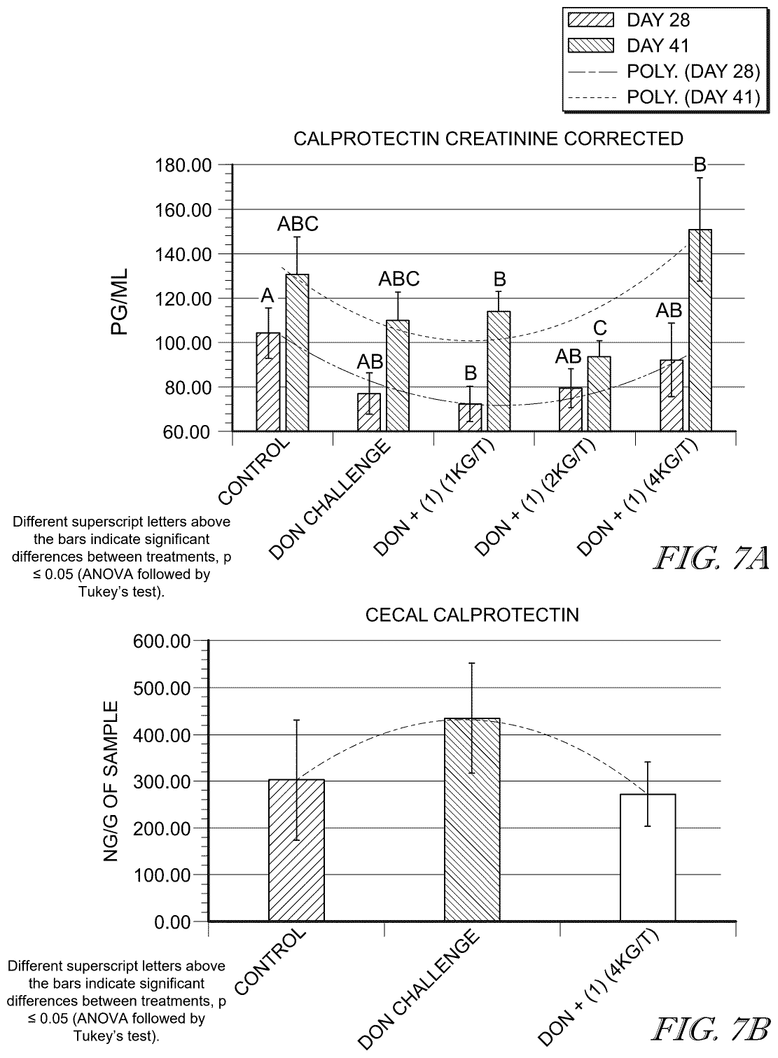

Conversely, the levels of calprotectin in caeca trended to be higher in the chicken receiving the challenge diet only and lower in the control when composition (1) was present (see FIGS. 7A and 7B). As a result of intestinal inflammation, neutrophilic white blood cells move in large numbers to the affected tissue. Once there, events such as the generation of oxygen radicals and breakdown of neutrophils could happen. This process releases cytosolic components, which contain various hydrolytic and proteolytic enzymes, along with calprotectin. In poultry, elevated levels of calprotectin in fecal or intestinal digesta samples serve as a marker for local inflammation. No changes in other biomarkers of inflammation were seen, such as in the concentrations of fibronectin.

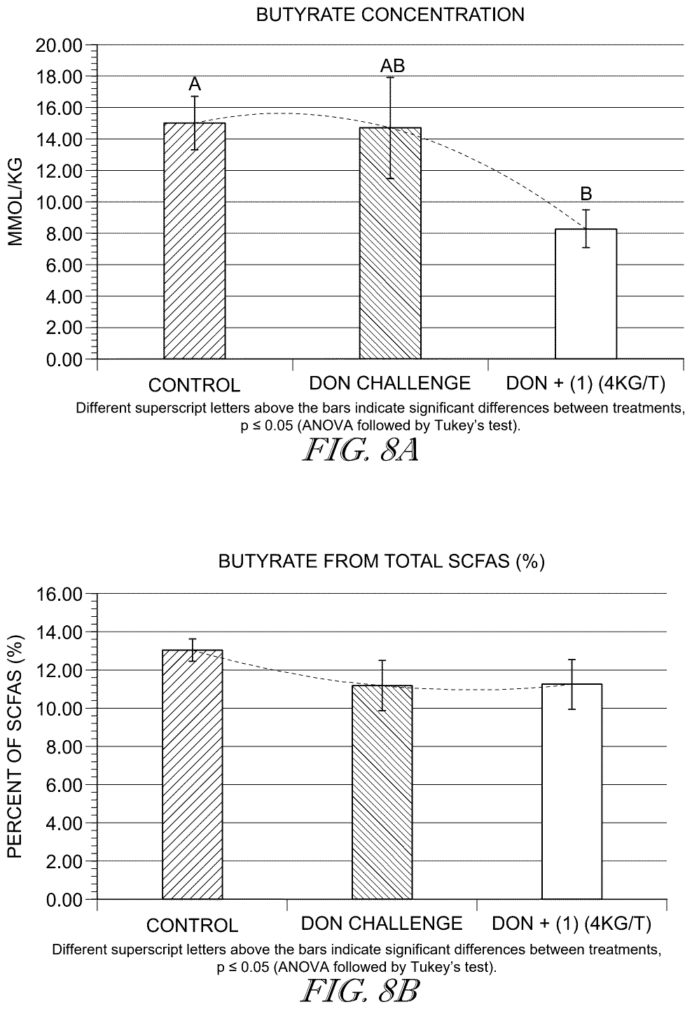

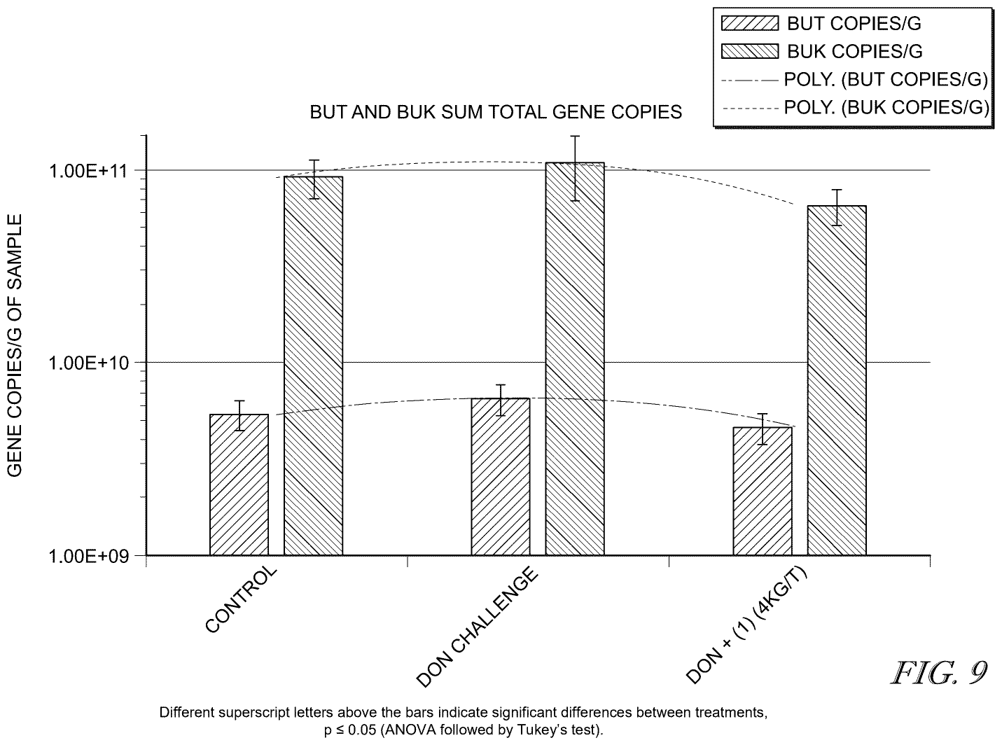

Ceacal samples also demonstrated a change in butyrate production with the inventive composition (see FIGS. 8A and 8B). Evaluated at the 4 kg/T inclusion rate, the butyrate concentration significantly decreased with composition (1), but its overall proportion from the total volatile fatty acid production remained unchanged, as well as the butyrate kinase (BUK) and butyryl-CoA acetate transferase (BUT) encoding genes (see FIG. 9). Additionally, total eubacterial population tended to increase with the contaminated diet compared to the control treatment, while the inventive composition maintained that population comparable to the control.

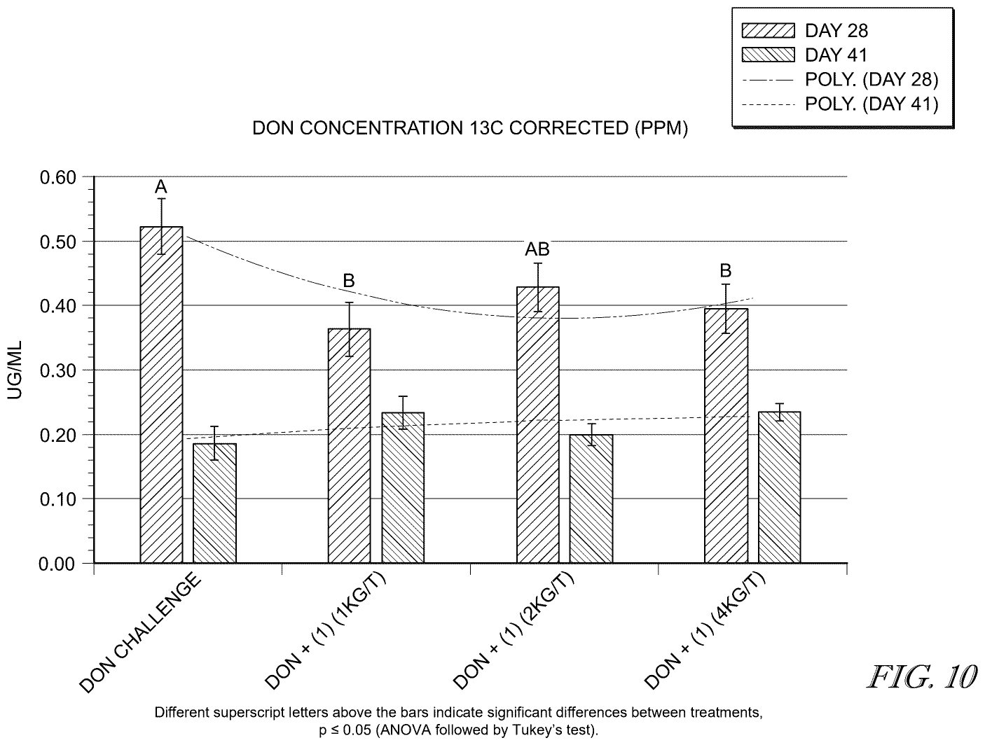

The quantification of the blood samples collected on VAMS for mycotoxins, established that deoxynivalenol concentrations, corrected using isotope dilution, showed a significant decrease for samples collected at Day 28 with the use of the inventive composition (see FIG. 10), though no significant differences were found between the three different inclusion rates (1, 2, and 4 kg/T) of composition (1). No significant differences were seen for the samples collected at Day 41 for deoxynivalenol levels, but DON concentrations measured where 2 to 3 times lower at Day 41 compared to Day 28 collection timepoints.

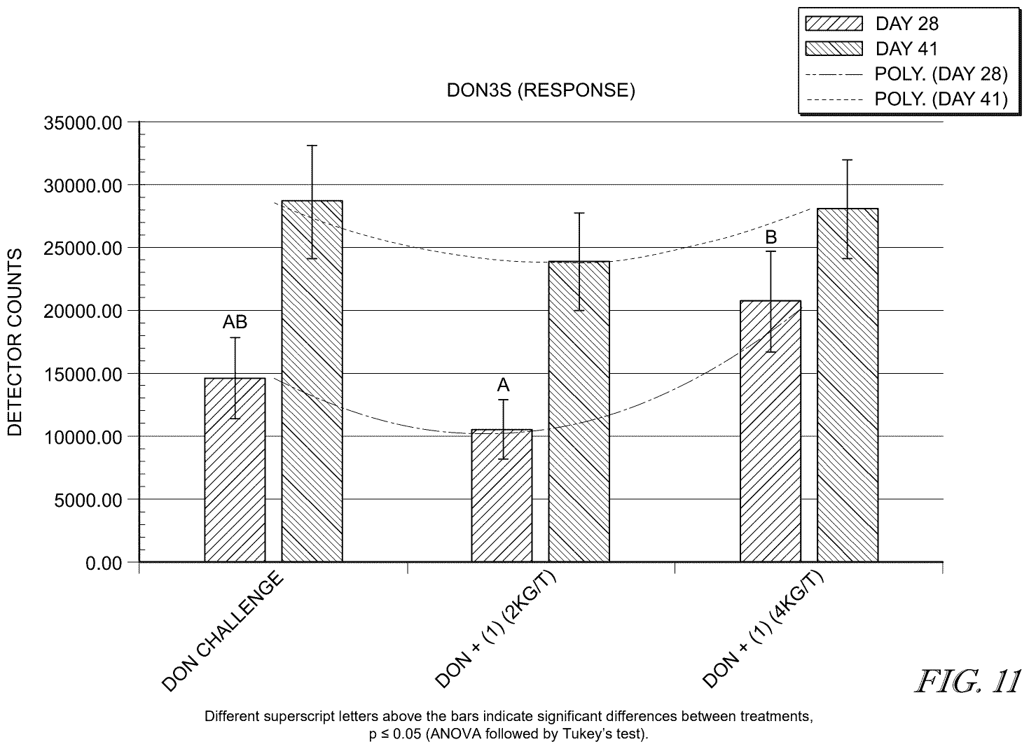

Other mycotoxin metabolites were found, such as the phase II conjugated metabolite, deoxynivalenol-sulfate (DON-sulfate or DON3S), but no significant differences were seen for its relative quantification (see FIG. 11). A trend toward lower or higher levels were seen for respectively 2.0 and 4.0 kg/T inclusion of the inventive composition. As a metabolite from the animal detoxification metabolism pathway, lower levels of this metabolite (i.e., deoxynivalenol-sulfate or DON3S) could indicate lower levels of DON parent molecules. Conversely, higher levels of the conjugated metabolite could also signify an increase of the metabolic processing of the DON molecule into DON-sulfate. These results demonstrated the efficacy of the yeast cell wall extract and clay material admixed to bacterial biomass rich in proteins comprised by composition (1), when incorporated in the diet of animals, at decreasing the amount of deoxynivalenol that can transfer through the intestinal barrier into the blood of broiler chickens fed a daily concentration of deoxynivalenol of around 8.0 μg/kg of feed.

As such, the present examples providing in vitro and in vivo data demonstrate the unexpected technical effects and synergistic results of the present composition comprising a yeast cell wall extract and clay material when admixed to a proteinaceous and/or cell wall extract(s) from bacterial fermentation from amino acid production, with or without an algal material, when the composition is provided to be in contact with an animal, including its cells and/or body parts (e.g., the gut or digestive tract). More specifically, the present data supports the significantly improved and/or increased ability of the present composition over traditional or historical compositions to sequester and/or absorb mycotoxins (e.g., external to an animal or internally in the gut of an animal) in order to prevent their migration, uptake, and/or absorption by the animal (e.g., in the bloodstream of the animal), which ultimately reduces and/or prevents contamination, infection, and/or destabilization of an animal or a human.

### Definitions

As used herein, the terms “absorb” or “absorption” refer to the process by which a material “takes in” or “sucks up” another substance. For example, “absorption” may refer to the process of absorbing or assimilating or uptaking substances into cells or across the tissues and organs through diffusion or osmosis (e.g. absorption of nutrients by the digestive system or absorption of drugs into the blood stream).

As used herein, the terms “administration” and “administering” refer to the act of giving a substance, including a drug, prodrug, or other agent, or therapeutic treatment to a subject (e.g., a subject or in vivo, in vitro, or ex vivo cells, tissues, and organs). Exemplary routes of administration can be through the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “admix” or “admixture” refers to the deliberate and/or controlled addition of one substance to another to modify its properties or the overall properties of the resulting composition or substance, and emphasizes the act of adding an extra component to another, as compared to “mixing,” which may occur by a variety of methods, including stirring, shaking, blending, folding, etc.

As used herein, the terms “adsorb” and “adsorption” refer to a process that occurs when a material is sequestered by, and/or accumulates on the surface of a composition (sequestrant and/or adsorbent), or a process in which a composition (e.g. yeast cell wall extract) binds to a target molecule (e.g. mycotoxins) in a sample thereby removing the target molecule from the sample.

As used herein, the terms “algae,” and the phrases “algal cells” and “algal material” refer to a group of predominantly aquatic, photosynthetic, and eukaryotic organisms of the kingdom of Protista, with cell features not found amongst plants and animals. Algae also comprise some prokaryotic organisms that lack a nucleus, such as cyanobacteria. The taxonomy of algae is contentious and subject to changes. Algae organisms can be placed in different divisions such as Chlorophyceae (green algae), Chromophyta, Chriptophyta, Rhodophyta (red algae), Dinoflagellata (Pyrrophyta), Euglenophyta, encompassing different classes such as Chlorophyceae, Bacillariophyceae (diatoms), Chrysophyceae (golden algae), Xanthophyceae, Cryptophyceae, Euglininophyceae, Cyanophyceae. Those organisms can be organized by having a single cell or multiple cells, and categorized as microorganisms or macroorganisms.

As used herein, the term “analyte” refers to an atom, a molecule, a substance or a chemical constituent. An analyte, in and of itself cannot be measured, rather, aspects or properties (physical, chemical, biological, etc.) of the analyte can be determined by using analytical procedures, such as for example HPLC. Likewise, one cannot measure a mycotoxin but can measure the mycotoxin fluorescence or UV absorption that is related to its concentration.

As used herein, the term “animal” refers to those of animal kingdom or Kingdom Animalia. This includes, but is not limited to livestock, farm animals, and/or pet animals. In some embodiments, an animal of the present disclosure is selected from avian, bovine, porcine, equine, ovine, and caprine, piscine, shellfish, camelids, feline, canine, and rodent species. In other embodiments, an animal of the present disclosure may include one or more of a cow, a bull, cattle, a pig, a swine, a sow, a chicken, a broiler, and/or a human. In other embodiments, the animal may comprise any internal or external portion or parts of the body of an animal, including the skin, one or more organs, such as the brain, the heart, the gut, the digestive system or digestive tract, and/or one or more animal cells or various components or types of cells of an animal, etc.

As used herein, the term “autotrophic” refers to the classification of an organism that is able to produce its own carbon source, energy and nutrients by using light, water, an inorganic carbon source and other chemicals. An algae, which creates food by photosynthesis, is autotrophic.

As used herein, the phrase “bacterial biomass” refers to the mass or total number of microorganisms in a given area, an ecosystem, or a production system at any given time.

As used herein, the phrase “bacterial biomass rich in protein” refers to protein rich co-products obtained from a bacterial biomass production. This biomass could be involved in the production of amino acids, vitamins, organic acids, enzymes, proteins and/or their salts obtained by fermentation of, but not limited to, Bacillaceae, Corynebacteriaceae, Enterococcaceae, Lactobacilaceae, Bifidobactericae, and/or specifically Bacillus coagulans, Bacillus subtilis, Bacillus velezensis, Bacillus licheniformis, Bacillus smithii, Corynebacterium casei, Corynebacterium glutamicum, Corynebacterium melassecola, Ensifer adhaerens, Enterococcus faecium, Escherichia coli K12 and/or Lactobacilluseae on substrate/culture medium comprising, consisting essentially of, and/or consisting of a carbon source mostly of vegetal origin, a nitrogen source of vegetal or chemical origin, vitamins, and/or minerals. Additionally, bacterial protein from Corynebacterium glutamicum refers to a protein product or byproduct from the production of amino acids by a culture of Corynebacterium glutamicum on substrates of vegetal or chemical origin, ammonia, and/or mineral salts.

As used herein, the phrase “bacterial cell wall” refers to the heteropolysaccharide cell wall meshwork of a bacterial cell. (i.e., Corynebacterium) comprising a heteropolysaccharide meshwork cell wall composed of peptidoglycan; arabinan, galactan, chitin, mannan, glucan and combination thereof; mycolic acids, including but not limited to, trehalose monomycolate, trehalose dimycolate, diacyltrehalose, polyacyltrehalose, phthiocerol dimycocerosate; glycolipids such as, but not limited to, sulfoglycolipid, phosphatidyl-myo-inositol mannosides, lipomannan, lipoarabinomannan, mannosylated lipoarabinomannan; and/or S-layer glycoproteins.

As used herein, the phrase “bacterial cell wall extract” refers to the bacterial cell wall that has been ruptured or “lysed” (e.g., during a rupture and lysing stage) and separated from the soluble intracellular components of the bacterial cell and comprising some of the individual constituents or combination thereof, comprising peptidoglycan; arabinan, galactan, chitin, mannan, glucan and combination thereof; mycolic acids, including but not limited to, trehalose monomycolate, trehalose dimycolate, diacyltrehalose, polyacyltrehalose, phthiocerol dimycocerosate; glycolipids such as, but not limited to, sulfoglycolipid, phosphatidyl-myo-inositol mannosides, lipomannan, lipoarabinomannan, mannosylated lipoarabinomannan; and/or S-layer glycoproteins.

As used herein, the term “bioavailability” refers to the fraction of a molecule or component that is available to an organism or reaches the systemic circulation. When a molecule or component is administered intravenously, its bioavailability is 100%. However, when a molecule or component is administered via other routes (such as orally), its bioavailability decreases (due to incomplete absorption and first-pass metabolism).

As used herein, the term “biomass” refers to the total quantity of weight of organisms in a given area or volume, i.e., the total mass or weight of a yeast cells, bacteria, fungi or algae accumulated during propagation, cultivation or fermentation.

As used herein, the terms “caeca” or “caecal” refer to a bodily cavity with only one opening to prevent flow through of digesta, often creating a pouch or sac, at the beginning of the large intestines and/or between the small and large intestines of the digestive tract or system of an animal. Often, the caeca is located in the lower right-hand side of the abdomen.

As used herein, the term “carrier” refers to a delivery system for a feed supplement that improve uniformity, dilution, dispersibility, while being edible and safe for the animal. Carriers could be composed of, but not limited to, inorganic materials such as silica or chalk, charcoal, activated charcoal, or any processed grain by-products organic materials such as corn, gluten feed, wheat bran, soybean meal, rice hulls, fiber, beet pulp, distillers dried grains, etc.

As used herein, the term “cell” refers to an autonomous self-replicating unit that may exist as functional independent unit of life (as in the case of unicellular organism, e.g. yeast), or as sub-unit in a multicellular organism (such as in plants and animals) that is specialized into carrying out particular functions towards the cause of the organism as a whole. There are two distinct types of cells: prokaryotic cells and eukaryotic cells.

As used herein, the phrase “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “centrifugation” refers to separating molecules by size or density using centrifugal forces generated by a spinning rotor.

As used herein, the term “clay” refers to mineral clays, synthetic, organoclays and any mixture(s) thereof.

As used herein, the terms “co-administration” and “co-administering” refer to the simultaneous or combined administration of at least two efficacious agent(s) or therapies (e.g., co-mixture of yeast cell wall extract(s), a clay component(s), and an algal material(s), a proteinaceous and/or cell wall extract(s) from bacterial or fungal fermentation from amino acid production) to a subject and/or material (e.g., feedstuff). Co-administration of two or more agents or therapies can be concurrent, or a first agent/therapy can be administered prior to a second agent/therapy.

As used herein, the term “complex” refers to an entity formed by association between two or more separate entities (e.g. association between two or more entities wherein the entities are the same or different (e.g. same or different chemical species). The association may be via a covalent bond or a non-covalent bond (e.g. via van der Waals, electrostatic, charge interaction, hydrophobic interaction, dipole interaction, and/or hydrogen bonding forces.

As used herein, the phrase “concentrated modified yeast cell wall extract” as used herein refers to concentrated yeast cell wall extract derived from modified or altered yeast.

As used herein, the phrase “concentrated yeast cell wall extract” refers to yeast cell wall extract that is concentrated via one or more procedures (e.g., by drying, such as during a drying and concentrating stage). In another example, a concentrated yeast cell wall extract is a yeast cell wall preparation or yeast cell wall extract preparation that is purified by removal of non-yeast cell wall components.

As used herein, the term “concentration” refers to the amount of a substance per defined space. It is usually expressed in terms of mass per unit of volume. To dilute a solution, one must add more solvent. By contrast, to concentrate a solution, one must reduce the amount of solvent.

As used herein, the phrases “cultivate yeast” and “growing yeast” refer to the act of populating and/or propagating yeast.

As used herein, the phrase “digestive system,” also referred to as “the gut,” refers to a system (including gastrointestinal system) in which digestion can occur in the body of an animal or human.

As used herein, the terms “digest” and “digestion” refer to the conversion of feedstuff into absorbable form; to soften, decompose, or break down by heat or moisture chemical or enzymatic action.

As referred herein, the terms “disease,” “infection,” and the phrase “pathological condition or response” refers to a state, signs, and/or symptoms that are associated with an impairment of the of the normal state of living animal or any of its organs or tissues that interrupts or modifies the performance of normal functions and may be a response to environmental factors including mycotoxicosis.

As used herein, the term “drying” refers to spray drying, freeze drying, air drying, vacuum drying or any other kind of process that reduces or eliminates liquid in a substance.

As used herein, the phrase “dry free flowing powder” refers to a free-flowing dry powder.

As used herein, the phrase “electrospray ionization” and the term “ESI” refer to generation of ions in a sample prior to be analyzed by a mass spectrometer when introduced in a liquid-phase and after undergoing nebulization, desolvation through vacuum pumping stages and ion formation in a gas phase by protonation, deprotonation and/or adduct formation under heat and electric current.

As used herein, the term “emerging mycotoxins” are newer or lesser-known forms of mycotoxins that are not routinely identified or that are presently unregulated and may pose risks to animal and human health or the environment. These substances may lack established health standards or be associated with new sources or pathways of exposure to animals and humans.

As used herein, the term “eukaryote” refers to organisms whose cells are organized into complex structures enclosed within membranes. “Eukaryotes” are distinguishable from “prokaryotes.” The term “prokaryote” refers to organisms that lack a cell nucleus or other membrane-bound organelles. The term “eukaryote” refers to all organisms with cells that exhibit the typical characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals.

As used herein, the term “feed” or the phrase “animal feed” refers to any substance or product intended for oral feeding to and/or consumption by animals. The feed may be directly and/or indirectly fed to or eaten by the animals, such that that the feed is intended to enter the animal digestive system, including the gut, and to be digested by the animal for nutritional and/or health purposes.

As used herein, the terms “feedstuff” and/or feedstuffs” refer to one or more material(s) or specific ingredients that make up a feed that is consumed by an animal. Examples of a feedstuff and/or feedstuffs include but are not limited to grains, forage, grass, and hay.

As used herein, the term “fermentation” refers to the biochemical process in which microorganisms, such as yeast, fungi, bacteria, or a mixture or combination thereof, convert carbohydrate sources under anaerobic conditions into biomass and for the production of various fermentation products, including acids, gases, alcohol, proteins, lipids, and other biological molecules. Fermentation is commonly conducted in a liquid medium (e.g., for the production of alcoholic beverages). This approach facilitates efficient management of fermentation conditions, such as agitation, temperature, pH, substrate composition, etc. Alternatively, fermentation can be conducted under solid-state conditions, where microorganisms are grown on solid substrates (e.g., grains, agricultural wastes, etc.) and utilized for producing fermented foods, enzymes, and bioactive compounds, which allows for the concentration of fermentation byproducts within the solid matrix.

As used herein, the term “formulation” refers to the process of creating a specific mixture or composition by combining various ingredients or components in a precise manner to achieve desired physical or chemical properties, and/or a specific function or performance or efficacy.

As used herein, the term “freeze-drying” and the term “lyophilization” and the term “cryodesiccation” refer to the removal of a solvent from matter in a frozen state by sublimation. This is accomplished by freezing the material to be dried below its eutectic point and then providing the latent heat of sublimation. Precise control of heat input permits drying from the frozen state without product melt-back. In practical applications, the process is accelerated and precisely controlled under reduced pressure conditions.

As used herein, the term “grinding” refers to reducing particle size by impact, shearing, or attrition.

As used herein, the phrase “the gut” refers to the “digestive system” or digestive tract, which is typically a tube that extends from the mouth to the anus of an animal or human. The gut plays a crucial role in the physical processing (e.g., digestion, absorption, adsorption, etc.) of food, feed, and/or feedstuffs as well as the nutrients and chemicals comprised therein. Typically, the gut includes several regions such as the esophagus, stomach, small and large intestines, as well as the rectum. In some embodiments of the present disclosure, the gut refers to the stomach and/or intestines.

As used herein, the term “heterotrophic” refers to a production process, often using fermentation and an absence of light, that relies on the consumption by a microorganism or macroorganism of organic energy and carbon sources for biomass productivity or optimal/maximal cell density, and/or by-product production of lipids, carbohydrates, pigments, nutrients, etc.

As used herein, the phrase “hydrolyzed yeast” refers to a specific preparation of yeast that is inactivated through autolysis governed by endogenous enzymes, chemical hydrolysis, or exogenous enzymatic hydrolysis process, which breakdowns the yeast cell wall and fragments internal constituents of the yeast cell, such as proteins or nucleic acids.

As used herein, the term “interlace” refers to two molecular entities that have a connection, lacing together, interweaving, integration or interspersing. In other words, one molecular structure becomes integrated into or within another molecular structure.

As used herein, the term “iso-mixture” is used in various scientific and engineering contexts to describe a mixture of substances that share certain characteristics or properties, typically involving a homogeneity, a similarity in properties, or application, e.g. an identical concentration for each of the mycotoxins in a mixture.

As used herein, the term “in vitro” refers to an artificial environment outside the living organism and to biological processes or reactions that would normally occur in an artificial environment. In vitro environments can comprise of, but are not limited to, test tubes and cell cultures.

As used herein, the term “in vivo” refers to studies and/or experiments conducted within a living organism, occurring within a biological organism.

As us herein, the phrase “mass spectrometry” refers to an analytical tool that is used to detect an analyte, generally an ion positively or negatively charged, according to its mass-to-charge ratio (m/z) present in a sample and detected using a scanning analyzer. The results are presented in the form of a mass spectrum, a plot of intensity as a function to mass-to-charge ratio.

As used herein, the phrase “mineral clay” refers to a naturally occurring or synthetic material composed primarily of fine-grained minerals (silicates) that show plasticity through a variable range of water content (which may be a result of water trapped in the structure by polar attraction) and can be hardened when dried and/or fired. Examples of silicates include, but are not limited to, phyllosilicate, bentonite, zeolite, aluminosilicate, montmorillonite, smectite, and kaolinite.

As referred herein, the term “mitigation” refers to the action of reducing the severity, impact, or negative effects of something undesirable (e.g. mycotoxins). Mitigation involves measures taken to minimize or alleviate potential harm, risks, or consequences and prevent or lessen the adverse effects of mycotoxins when present in animal feed.

As used herein, the phrase “modified yeast cell wall” refers to yeast cell wall of modified or altered yeast.

As used herein, the phrase “modified yeast cell wall extract” refers to yeast cell wall extract of modified or altered yeast.

As used herein, the phrases “modified clay” and “organoclay” refer to an organically modified phyllosilicate, derived from a naturally occurring clay mineral. By exchanging the original interlayer cations for organocations (typically quaternary alkylammonium ions) or polysaccharides, an organophilic surface is generated, consisting of covalently linked organic moieties. The lamellar structure remains analogous to the parent phyllosilicate.

As used herein, the term “mycotoxicosis” refers to a condition in which mycotoxins pass the resistance barriers of the human or animal body. Mycotoxicosis can be considered either an infection or a disease and may have a deleterious effect on those afflicted.

As used herein, the term “mycotoxin” refers to a toxic and or carcinogenic compound(s) produced by various fungal species (e.g. deoxynivalenol produced by fungus Fusarium graminearum).

As used herein, the phrase “masked mycotoxins” encompasses only conjugated forms of mycotoxins produced by the plant metabolic activity.

As used herein, the phrase “parietal components” refers to component in the yeast, algae or bacteria microorganisms that are constituents of their respective cell wall.

As used herein, the term “pellet(s)” refers to a type of animal feed that is typically created using compression of the ingredients into small pieces, typically in a round or cylindrical shape. Pelleting of animal feed is believed to improved storage life and ease of handling, as well as improved digestibility when the animal feed pellets are consumed by animals.

As used herein, the phrase “pharmaceutical composition” refers to the combination of an active agent (e.g., a composition comprising a viable yeast cell, yeast cell wall, or modified yeast cell wall component of the invention) with a carrier, inert or active, making the composition especially suitable for diagnostic or therapeutic use in vitro, in vivo or ex vivo.

As used herein, the phrases “pharmaceutically acceptable” and “pharmacologically acceptable” refer to compositions that do not substantially produce more known adverse reactions than known beneficial reactions.

As used herein, the term “photosynthesis” refers to a system of biological processes by which green plants and organisms, such as an algae, transform light energy, carbon dioxide and water into chemical energy to produce organic compounds used as a carbon source.

As used herein, the term “purified” or the phrase “to purify” refers to the removal of foreign components from the sample. When used in a chemical context “purified” or “to purify” refers to physical separation of a chemical substance of interest from the undesired or contaminated substances. Commonly used methods for purification of organic molecules include, but are not limited to the following: mechanical filtration, affinity purification, centrifugation, evaporation, extraction of impurity, crystallization, adsorption, distillation, sublimation, smelting, refining, electrolysis and dialysis.

As used herein, the term “sample” is used in a broad sense to include a specimen from any source, as well as synthetic, biological or environmental samples. Biological samples can be obtained from animals or plants and encompass fluids, solids, tissues and gases. Environmental samples include environmental material such as surface matter, soil, water and industrial samples.

As used herein, the terms “sequester” and/or “sequestration” refer to physical association (e.g. hydrogen bonding, ionic bonding, covalent bonding or other type of bonding) of two or more entities that came in contact with one another (e.g. forming a complex). Exemplary forms of associations include, but are not limited to, hydrogen bonding, coordination, and ion par formation. Sequestration interactions might involve a variable number of chemical interactions (e.g. chemical bonds) depending on the stereochemistry and geometry of each entity (e.g. further defining the specificity of sequestration). When two or more entities are sequestered by way of chemical bonds, but might also be associated via charge, dipole-dipole or other types of interactions.

As used herein, the phrases “sequestration agent” and/or “sequestering agent” refers to an entity that is capable forming a complex with a second entity. It refers to the docking or the encagement of two or more molecular entities that come into contact with one another and form a stable complex. Typical forms of docking or encagement are by hydrogen bonding, coordination, and ion pair formation. The interaction involves a variable number of chemical interactions depending on the stereochemistry and geometry of each molecular entity which further defines the specificity of the sequestration. Often, “bonding” is used to describe the sequestration of molecular entities; however, the term “bonding” is not a technically accurate way of describing the interaction. When two molecular entities are sequestered they may be sequestered by way of “binding” however, they may also be sequestered through other interactions.

As used herein, the term “signal” is used generally in reference to any detectable process which indicates that a reaction has occurred. Signals can be assessed quantitatively as well as qualitatively. Examples of types of “signals” include, but are not limited to, radioactive signals, fluorometric signals or colorimetric product/reagent signals.

As used herein, the term “sorption” refers to both adsorption and absorption.

As used herein, the phrase “spray drying” refers to a commonly used method of drying a substance containing liquid using hot gas to evaporate the liquid to reduce or eliminate liquid in the substance. In other words, the material is dried by spraying or atomizing into a draft of heated dry air.

As used herein, the term “treatment” refers to the improvement and/or reversal of the symptoms of disease (e.g., mycotoxicosis). The term “treatment” refers to both therapeutic treatment and prophylactic or preventative measures. For example, subjects that may benefit from treatment with compositions and methods of the present invention include those already with a disease and/or disorder (e.g., mycotoxicosis) as well as those in which a disease and/or disorder is to be prevented (e.g., using a prophylactic treatment of the present invention).

As referred herein, the term “toxic” refers to any detrimental, deleterious, harmful, or otherwise negative effect(s) on an animal a cell, or a tissue as compared to the same cell or tissue prior to the contact with toxin.

As used herein, the term “ultra-,” the phrase “high-performance liquid chromatography,” and the terms “UPLC” or “HPLC” refer to a form of liquid chromatography to separate compounds. The compounds are dissolved in a solution. Compounds are separated by injecting the sample mixture into a column, through a solvent or a solvent mixture flowing to elute components of the mixture from the column. UPLC or HPLC instruments comprise a reservoir of a mobile phase, a pump, an injector, a separation column and a detector. The presence of analytes in the column effluent is recorded by quantitative detection of a change in a refractive index, UV-VIS absorption at a set wavelength, a fluorescence, after excitation with suitable wavelength, or after ionization by mass spectrometry.

As used herein, the term “yeast” and the phrase “yeast cells” refer to eukaryotic microorganisms classified in the kingdom Fungi, having a cell wall, cell membrane, and intracellular components. Yeasts do not form a specific taxonomic or phylogenetic grouping. Currently about 1,500 species of yeasts are known. It is estimated that only 1% of all yeast species have been described.

The term “yeast” is often taken as a synonym for S. cerevisiae, but the phylogenetic diversity of yeasts is shown by their placement in both divisions Ascomycota and Basidiomycota. The budding yeasts (“true yeasts”) are classified in the order Saccharomycesles. Most species of yeast reproduce asexually by budding, although some reproduce by binary fission. Yeasts are unicellular, although some species become multicellular through the formation of a string of connected budding cells known as pseudohyphae, or false hyphae. Yeast size can vary greatly depending on the species, typically measuring 3-4 μm in diameter, although some yeast can reach over 40 μm.

As used herein, the phrase “yeast-based” refers to a product that contains yeast or yeast constituents, i.e., yeast cell wall, yeast cell wall extract . . . .

As used herein, the phrase “yeast cell wall,” also referred to as “YCW,” refers to the cell wall of a yeast organism that surrounds the plasmic membrane and the intracellular components of the yeast. Yeast cell wall includes both the outer layer (mainly mannan) and the inner layer (mainly glucan and chitin) of the yeast cell wall. A function of the cell wall is to provide structure and protect the yeast interior (its metabolic activity center). Signaling and recognition pathways take place in the yeast cell wall. The composition of yeast cell wall varies from strain to strain and according to growth conditions of yeast.

As used herein, the phrase “yeast cell wall extract,” also referred to as “YCWE”, refers to the yeast cell wall of yeast that has been ruptured or “lysed” (e.g., during a rupture and lysing stage) and separated from the soluble intracellular components of the yeast cell.

As used herein, the phrases “yeast glucomannan(s),” “glucomannan yeast product,” “β-glucans,” “glucans,” and/or “mannans” refer to the carbohydrate constituents forming the yeast cell wall molecular network. β-glucans are long chains of glucose molecules linked together through carbon 1,3 and 1,6 linkages, and they play a crucial role in maintaining the cell wall strength and elasticity. They form the inner layer of the yeast cell wall. Mannans are complex carbohydrates composed of mannose important for cell wall architecture and are often attached to proteins, forming glycoproteins and the outer layer of the yeast cell wall. Chitin is another polymer present in the most inner part of the yeast cell wall, made from N-acetylglucosamine, a derivative of glucose. Chitin is present in smaller amounts compared to β-glucans and mannans but plays an important role in maintaining the structural integrity of the cell wall.

As used herein, the phrase “yeast preparation” refers to the process of preparing yeast for use in a fermentation process, converting sugars and other carbohydrates into organic acids, amino acids, alcohols, gases, proteins, vitamins, nutrients, and other byproducts. Baker's yeasts, brewer's yeasts and distiller's yeast are yeast preparations that may be utilized in the present compositions or methods that involve different yeast strains, as well as different biotic and abiotic conditions under spontaneous or controlled fermentation processes.

As used herein, the phrase “yeast supplement” refers to dietary and nutritional supplements made from active, inactive, or deactivated yeast and contains some levels of vitamins, minerals, antioxidants, other nutrients alongside proteins, carbohydrates, fibers, and lipids.

Moreover, the features illustrated or described in connection with one exemplary embodiment may be combined with any other feature or element of any other embodiment described herein. Such modifications and variations are intended to be included within the scope of the present disclosure. Further, a person skilled in the art will recognize that terms commonly known to those skilled in the art may be used interchangeably herein.

The above embodiments are described in sufficient detail to enable those skilled in the art to practice what is claimed and it is to be understood that logical, mechanical, and electrical changes may be made without departing from the spirit and scope of the claims. The detailed description is, therefore, not to be taken in a limiting sense.

As used herein, an element or step recited in the singular and proceeded with the word “a” or “an” should be understood as not excluding plural of said elements or steps, unless such exclusion is explicitly stated. Furthermore, references to “one embodiment” of the presently described subject matter are not intended to be interpreted as excluding the existence of additional embodiments that also incorporate the recited features. Specified numerical ranges of units, measurements, and/or values comprise, consist essentially or, or consist of all the numerical values, units, measurements, and/or ranges including or within those ranges and/or endpoints, whether those numerical values, units, measurements, and/or ranges are explicitly specified in the present disclosure or not.

Unless defined otherwise, technical, and scientific terms used herein have the same meaning as is commonly understood by one of ordinary skill in the art to which this disclosure belongs. The terms “first,” “second,” “third” and the like, as used herein do not denote any order or importance, but rather are used to distinguish one element from another. The term “or” is meant to be inclusive and mean either or all of the listed items. In addition, the terms “connected” and “coupled” are not restricted to physical or mechanical connections or couplings, and can include electrical connections or couplings, whether direct or indirect.

Moreover, unless explicitly stated to the contrary, embodiments “comprising,” “including,” or “having” an element or a plurality of elements having a particular property may include additional such elements not having that property. The term “comprising” or “comprises” refers to a composition, compound, formulation, or method that is inclusive and does not exclude additional elements, components, and/or method steps. The term “comprising” also refers to a composition, compound, formulation, or method embodiment of the present disclosure that is inclusive and does not exclude additional elements, components, or method steps.

The phrase “consisting of” or “consists of” refers to a compound, composition, formulation, or method that excludes the presence of any additional elements, components, or method steps. The term “consisting of” also refers to a compound, composition, formulation, or method of the present disclosure that excludes the presence of any additional elements, components, or method steps.

The phrase “consisting essentially of” or “consists essentially of” refers to a composition, compound, formulation, or method that is inclusive of additional elements, components, or method steps that do not materially affect the characteristic(s) of the composition, compound, formulation, or method. The phrase “consisting essentially of” also refers to a composition, compound, formulation, or method of the present disclosure that is inclusive of additional elements, components, or method steps that do not materially affect the characteristic(s) of the composition, compound, formulation, or method steps.

Approximating language, as used herein throughout the specification and claims, may be applied to modify any quantitative representation that could permissibly vary without resulting in a change in the basic function to which it is related. Accordingly, a value modified by a term or terms, such as “about,” and “substantially” is not to be limited to the precise value specified. In some instances, the approximating language may correspond to the precision of an instrument for measuring the value. Here and throughout the specification and claims, range limitations may be combined and/or interchanged. Such ranges are identified and include all the sub-ranges contained therein unless context or language indicates otherwise.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances, the modified term may sometimes not be appropriate, capable, or suitable.

It is to be understood that the above description is intended to be illustrative, and not restrictive. For example, the above-described embodiments (and/or aspects thereof) may be used individually, together, or in combination with each other. In addition, many modifications may be made to adapt a particular situation or material to the teachings of the subject matter set forth herein without departing from its scope. While the dimensions and types of materials described herein are intended to define the parameters of the disclosed subject matter, they are by no means limiting and are exemplary embodiments. Many other embodiments will be apparent to those of skill in the art upon reviewing the above description. The scope of the subject matter described herein should, therefore, be determined with reference to the appended claims, along with the full scope of equivalents to which such claims are entitled.

This written description uses examples to disclose several embodiments of the subject matter set forth herein, including the best mode, and also to enable a person of ordinary skill in the art to practice the embodiments of disclosed subject matter, including making and using the devices or systems and performing the methods. The patentable scope of the subject matter described herein is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

While only certain features of the invention have been illustrated and described herein, many modifications and changes will occur to those skilled in the art. It is, therefore, to be understood that the appended claims are intended to cover all such modifications and changes as fall within the true spirit of the invention.

What is claimed is:

1. A co-mixture composition configured to interact with a mycotoxin, comprising:
a. a yeast cell, including a yeast cell wall or extract therefrom, having about 20 wt % to about 99 wt % of the composition and
b. a clay material having about 0.1 wt % to about 10 wt % of the composition, admixed to
c. a proteinaceous or cell wall extract from a bacterial fermentation process or a fungal fermentation process having about 0.5 wt % to about 99 wt % of the composition.

2. The co-mixture composition of claim 1, further comprising an algal material having about 1 wt % to about 99 wt % of the composition.

3. The co-mixture composition of claim 1, wherein the yeast cell is selected from the group consisting of Saccharomyces, Candida, Kluyveromyces, Torulaspora, and a combination thereof.

4. The co-mixture composition of claim 1, wherein the clay material is a zeolite, a bentonite, an aluminosilicate, a montmorillonite, a smectite, a kaolinite, an organoclay, a modified clay, or mixtures thereof.

5. The co-mixture composition of claim 1, wherein the proteinaceous and/or cell wall extract(s) is selected from a bacterial strain selected from the group consisting of the genus Corynebacterium, Brevibacterium, Escherichia coli, Enterobacter, Lactobacillus, Pseudomonas, and Bacillus or a fungal strain selected from the group consisting of the genus Aspergillus, Candida, Fusarium, and Saccharomyces that is used in an industrial fermentation process.

6. The co-mixture composition of claim 1, wherein the proteinaceous or cell wall extract from the bacterial fermentation process or the fungal fermentation process is dried.

7. The co-mixture composition of claim 2, wherein the algal material belongs to the algae cell division Chlorophyceae (green algae), Chromophyta, Chriptophyta, Rhodophyta (red algae), Dinoflagellata (Pyrrophyta), Euglenophyta or from Chlorella, Asterarcys quadricellulare, Aurantiochytrium, and Schizochytrium species.

8. The co-mixture composition of claim 1, wherein the mycotoxin is selected from the group consisting of aflatoxins, ochratoxins, fumonisins, emerging Fusarium mycotoxins, Aspergillus mycotoxins, Penicillium mycotoxins, zearalenone, ergot alkaloids mycotoxins, AAL toxins, acetoxyscirpenediol, acetyldeoxynivalenol, acetylneosolaniol, acetyl T-2 toxin, acetyl HT-2, aflatoxins including aflatoxin B1 and B2 and G1 and G2, aflatoxicol, aflatrem, altenuic acid, alternariol, altertoxin, altersolanols, Alternaria toxins, apicidins, arugosins, asperazines, aspergillic acid, aspergillumarins, asperlicins, aspewentins, aspochalasins, aurofusarin, aurosperones, aurovertins, austalides, austdiol, austamide, austocystin, avenacein, baccharinoids, beauvericin, bentenolide, brevianamide, butenolide, calonectrin, chaetoglobosin, chevalones, citrinin, citreoviridin, citreoviridinol, cochliodinol, coniochaetons, cytochalasins, cyclosporins, cytochalasins, cyclopiazonic acid, deacetylcalonectrin, decarestrictine, deoxynivalenol, diacetoxyscirpenol, diacetyldeoxynivalenol, destruxins A and B, elymoclavines, enniatins such as enniatins A/A1 and B/B1, ergot toxins and endophytes such as ergine, ergocornine, ergocristine, ergocryptine, ergometrine, ergonine, ergosine, ergotamine, ergovaline, lysergol, lysergic acid, methylergonovine, and related epimers, fructigenines, fumigaclavines, fumagillin, fumiquinazolines, fumitremorgins, fumonisins including fumonisin A1 and B1 and B2 and B3, fusarenon X, fusaric acid, fusarin, fusarielin, fuscofusarin, geodin, geomycins, gliotoxin, griseophenones, griseofulvin, HT-2 toxin, ipomeanine, islanditoxin, isofumigaclavines A and B lateritin, leporisines, lolitrems, lycomarasmine, malformins, marcfortines, maleagrins, maltoryzine, miophytocens, moniliformin, monoacetoxyscirpenol, mycophenolic acid, neosolaniol, nigerapyrones, nivalenol, nordeodeoxynivalenol, NT-1 toxin, NT-2 toxin, ochratoxins such as ochratoxins A and B, oxalic acid, paraherquamide, paspalines, paspalitrems A and B, patulin, paxilline, penicillenol, penicillic acid, penitrems such as penitrem A, phomopsins, PR-toxin, psychrophilins, pyripyropenes, roridins, roritoxins, roquefortines such as roquefortine C, rubratoxin, rubroskyrin, rubrosulphin, rugulosin, satratoxins, scirpentriol, slaframine, solaniol, sporotrichiol, stephacisins, sterigmatocystin, sulochrin, swainsonine, T-2 toxin, tentoxin, terreins, territrems, tetrahydroaltersolanols, triacetoxyscirpendiol, trichothecenes, trichodermin, trichothecin, trichoverrins, trichoverrols, tryptoquivalene, verrucarins, versicolorins, versiconols, verruculogen, viopurpurin, viomellein, viriditoxin, wortmannin, xanthocillin, xanthomegnin, yavanicin, zanones, zearalenols, zearalanones, zearalenone and subfamilies, and/or possible conjugates and metabolites of the aforementioned mycotoxins, and combinations thereof.

9. The co-mixture composition of claim 1, wherein the composition has an average adsorption rate for one or more mycotoxins ranging from about 33% to about 100%.

10. The co-mixture composition of claim 1, wherein at least a portion of the composition has an improved efficacy to sequester or adsorb one or more mycotoxins over traditional compositions.

11. The co-mixture composition of claim 10, wherein the composition has an increased average adsorption rate for deoxynivalenol (DON) of about 211% or for fusaric acid of about 285%.

12. The co-mixture composition of claim 1, wherein the composition does not reduce its ability to interact with the mycotoxin when ground, dried, spray dried, vacuum dried, or heated.

13. A method of sequestering one or more mycotoxins in an animal, comprising:
a. providing to the animal for feeding or consumption, a composition comprising:
i. a yeast cell, including a yeast cell wall or extract therefrom, having about 20 wt % to about 99 wt % of the composition,
ii. a clay material having about 0.1 wt % to about 10 wt % of the composition,
iii. an optional algal material having about 0 wt % to about 10 wt %, admixed to
iv. a proteinaceous or cell wall extract from a bacterial fermentation process or a fungal fermentation process having about 0.5 wt % to about 99 wt % of the composition, and
v. an optional carrier having about 0 wt % to about 50 wt % of the composition,
b. increasing sequestration of the one or more mycotoxins within the gut of the animal, and
c. reducing absorption of the one or more mycotoxins in the bloodstream of the animal.

14. The method of claim 13, wherein feeding the composition is in an amount ranging from about 2 g/animal/day to about 50 g/animal/day.

15. The method of claim 13, wherein consumption of the composition by the animal further comprises providing the composition with an organic material selected from the group consisting of an animal feedstuff, a liquid, a water, an animal bedding and an animal clothing.

16. The method of claim 15, wherein providing the composition with the animal feedstuff comprises the composition being at about 0.0125 wt % to about 10 wt % of the animal feedstuff.

17. The method of claim 15, wherein providing the composition with the animal feedstuff comprises an inclusion rate of the composition at about 0.125 to about 4.0 kg/T of the animal feedstuff.

18. The method of claim 13, wherein grinding, drying, spray drying, vacuum drying, or heating the composition does not reduce its ability to sequester or adsorb the one or more mycotoxins.

19. A mycotoxin-sequestering composition configured to interact with an animal, comprising:
a. a yeast cell, including a yeast cell wall or extract therefrom, having about 70 wt % to about 90 wt % of the composition,
b. a clay material having about 0.1 wt % to about 4 wt % of the composition,
c. an algal material having about 1 wt % to about 10 wt % of the composition, all admixed to
d. a dried proteinaceous or cell wall extract from a bacterial fermentation process or a fungal fermentation process having about 10 wt % to about 30 wt % of the composition, and
e. a carrier having about 1 wt % to about 50 wt % of the composition,
wherein at least a portion of the mycotoxin-sequestering composition has an improved efficacy over traditional compositions to sequester or adsorb one or more mycotoxins present on or within the animal.

20. The mycotoxin-sequestering composition of claim 19, wherein the composition has an increased average adsorption rate for deoxynivalenol (DON) of about 211% or for fusaric acid of about 285% over traditional compositions.
